(19) United States
(12) Patent Application Publication
(10) Pub. No.: US 2010/0256392 A1
(43) Pub. Date: Oct. 7, 2010

(54) POLYMORPHS OF SUNITINIB BASE AND PROCESSES FOR PREPARATION THEREOF

(75) Inventors: Ales Gavenda, Lhotka (CZ); Ettore Bigatti, Rho (IT); Alexandr Jegorov, Dobra Voda (CZ); Augusto Canavesi, Locate Veresino (IT); Pavel Vraspir, Rymarov (CZ); Judith Aronhime, Rehovot (IL); Peter Lindsay MacDonald, Gentilino (CH); Francesca Scarpitta, Ivrea (IT); Marco Villa, Milano (IT); Paolo Angioletti, Lainate (IT)

Correspondence Address: MERCHANT & GOULD PC, P.O. BOX 2903, MINNEAPOLIS, MN, 55402-0903, US

(73) Assignee: TEVA PHARMACEUTICAL INDUSTRIES LTD., Petach-Tikva (IL)

(21) Appl. No.: 12/743,568

(22) PCT Filed: Nov. 21, 2008
(86) PCT No.: PCT/US08/84366

§ 371 Date: May 18, 2010

Related U.S. Application Data
(60) Provisional application No. 60/989,560, filed on Nov. 21, 2007.

(51) Int. Cl. C07D 403/06

(52) U.S. Cl. 548/468

(57) ABSTRACT

The present invention provides polymorphs of Sunitinib base and processes for preparation thereof.

### RELATED APPLICATIONS

This application claims the benefit of U.S. Provisional Application Nos. 60/989,560, filed Nov. 21, 2007; 61/030,167, filed Feb. 20, 2008; 61/042,138, filed Apr. 3, 2008; 61/045,196, filed Apr. 15, 2008; 61/048,467, Apr. 28, 2008; 61/058,053, Jun. 2, 2008; 61/061,069, filed Jun. 12, 2008; 61/061,920, filed Jun. 16, 2008; 61/078,650, filed Jul. 7, 2008; 61/082,405, filed Jul. 21, 2008; 61/108,078, filed Oct. 24, 2008; 61/031,773, filed Feb. 27, 2008; 61/041,439, filed Apr. 1, 2008; 61/048,460, filed Apr. 28, 2008; 61/058,417, filed Jun. 3, 2008; 61/059,088, filed Jun. 5, 2008; 61/084,156, filed Jul. 28, 2008; 61/087,859, filed Aug. 11, 2008; 61/101,527, filed Sep. 30, 2008; 61/059,222, filed Jun. 5, 2008; 61/085,991, filed Aug. 4, 2008; 61/088,554, Aug. 13, 2008, each of which is incorporated herein by reference in its entirety.

### FIELD OF INVENTION

The present invention relates to polymorphs of Sunitinib base and processes for preparation thereof.

### BACKGROUND OF THE INVENTION

Sunitinib base of the following formula

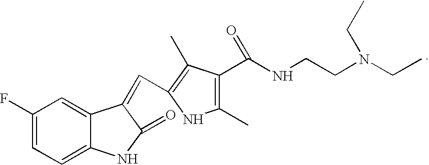

is an intermediate for Sunitinib salts, such as Sunitinib malate of the following formula.

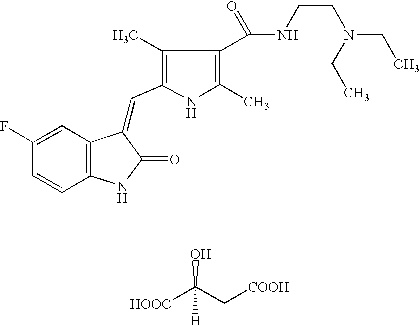

Sunitinib malate is marketed under the trade name Sutent® by Pfizer. It is an oral, multi-targeted tyrosine kinase inhibitor used for treatment of various types of cancer.

Sunitinib and salts thereof, process of preparation thereof and the use of these salts are disclosed in U.S. Pat. No. 6,573,293 B2.

The preparation of Sunitinib base that is disclosed in U.S. Pat. No. 6,573,293 is done by condensation of 5-formyl-2,4-1H-pyrrole-3-carboxylic acid (2-diethylaminoethyl)amide with 5-fluoro-1,3-dihydro-indol-2-one in EtOH in the presence of pyrrolidine; where Sunitinib base is isolated by filtration from the reaction mixture.

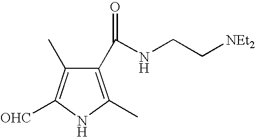

### 5 formyl 2,4 1H pyrrole 3 carboxylic acid (2 diethylaminoethyl)amide

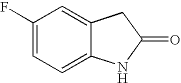

### 5 fluoro 1,3 dihydro indol 2 one

U.S. Pat. No. 7,119,209 also discloses the preparation of Sunitinib base by condensing 5-formyl-2,4-1H-pyrrole-3-carboxylic acid (2-diethylaminoethyl)amide with 5-fluoro-1,3-dihydro-indol-2-one in MeCN; where Sunitinib base is isolated by filtration from the reaction mixture.

The present invention relates to polymorphic forms of Sunitinib base.

Polymorphism, the occurrence of different crystal forms, is a property of some molecules and molecular complexes. A single molecule, like Sunitinib base, may give rise to a variety of solid state forms having distinct crystal structures and physical properties like melting point, x-ray diffraction pattern, infrared absorption fingerprint, and solid state NMR spectrum. One solid state form may give rise to thermal behavior different from that of another solid state form. Thermal behavior can be measured in the laboratory by such techniques as capillary melting point, thermogravimetric analysis (“TGA”), and differential scanning calorimetry (“DSC”), which have been used to distinguish polymorphic forms.

The difference in the physical properties of different solid state forms results from the orientation and intermolecular interactions of adjacent molecules or complexes in the bulk solid. Accordingly, polymorphs are distinct solids sharing the same molecular formula yet having distinct advantageous physical properties compared to other crystalline forms of the same compound or complex.

The discovery of new polymorphic forms of Sunitinib base provides a new opportunity to improve the performance of the synthesis of the active pharmaceutical ingredient (API), Sunitinib malate, by producing solid state forms of Sunitinib base having improved characteristics, such as flowability, and solubility. Thus, there is a need in the art for polymorphic forms of Sunitinib base.

### SUMMARY OF THE INVENTION

In one embodiment, the present invention encompasses anhydrous sunitinib base.

In another embodiment, the present invention encompasses crystalline Sunitinib base characterized by data selected from a group consisting of a PXRD pattern having any 5 peaks selected from the list consisting of: 4.5, 9.0, 15.1, 16.7, 18.3, 19.0, 20.4, 21.8, 25.9 and 27.4 deg±0.2 degrees 2-theta, a PXRD pattern as depicted in FIG. 1 and combination thereof.

In one embodiment, the present invention encompasses crystalline Sunitinib base characterized by data selected from a group consisting of a PXRD pattern having any 5 peaks selected from the list consisting of: 3.8, 7.8, 9.0, 10.2, 11.8, 15.8, 17.9, 20.3, 26.1 and 26.8 deg±0.2 degrees 2-theta, a PXRD pattern as depicted in FIG. 2 and combination thereof.

In one embodiment, the present invention encompasses crystalline Sunitinib base characterized by data selected from a group consisting of a PXRD pattern having any 5 peaks selected from the list consisting of: 5.0, 10.0, 13.0, 14.7, 16.0, 20.1, 21.9, 25.7, 26.6 and 28.3 deg±0.2 degrees 2-theta, a PXRD pattern as depicted in FIG. 5 and combination thereof.

In one embodiment, the present invention encompasses crystalline Sunitinib base characterized by data selected from a group consisting of a PXRD pattern having any 5 peaks selected from the list consisting of: 7.6, 9.3, 12.1, 15.2, 16.3, 19.2, 24.2, 25.5, 26.2 and 28.2 deg±0.2 degrees 2-theta, a PXRD pattern as depicted in FIG. 6.

In one embodiment, the present invention encompasses crystalline Sunitinib base characterized by data selected from a group consisting of a PXRD pattern having any 5 peaks selected from the list consisting of: 3.5, 5.9, 6.5, 8.3, 10.5, 11.3, 14.1, 17.9, 20.7, 26.0 and 27.9 deg±0.2 degrees 2-theta, a PXRD pattern as depicted in FIG. 7 and combination thereof.

In one embodiment, the present invention encompasses crystalline Sunitinib base characterized by data selected from a group consisting of a PXRD pattern having any 5 peaks selected from the list consisting of: 8.2, 10.9, 13.2, 13.6, 16.1, 18.8, 19.2, 20.9, 23.5, 26.1 and 29.6 deg±0.2 degrees 2-theta, a PXRD pattern as depicted in FIG. 8 and combination thereof.

In one embodiment, the present invention encompasses crystalline Sunitinib base characterized by data selected from a group consisting of a PXRD pattern having any 5 peaks selected from the list consisting of: 3.9, 7.8, 8.9, 9.2, 11.7, 13.9, 15.4, 16.0, 17.9, 20.4, 26.7 and 27.8 deg±0.2 degrees 2-theta, a PXRD pattern as depicted in FIG. 9 and combination thereof.

In one embodiment, the present invention encompasses crystalline Sunitinib base characterized by data selected from a group consisting of a PXRD pattern having any 5 peaks selected from the list consisting of: 3.8, 7.6, 8.5, 9.5, 10.4, 11.4, 16.5, 17.8, 20.6, and 27.0 deg±0.2 degrees 2-theta, a PXRD pattern as depicted in FIG. 10 and combination thereof.

In one embodiment, the present invention encompasses crystalline Sunitinib base characterized by data selected from a group consisting of a PXRD pattern having any 5 peaks selected from the list consisting of: 3.8, 7.6, 8.9, 9.3, 10.4, 17.8, 20.5, 21.7, 26.2, and 26.9 deg±0.2 degrees 2-theta, a PXRD pattern as depicted in FIG. 11 and combination thereof.

In one embodiment, the present invention encompasses crystalline Sunitinib base characterized by data selected from a group consisting of a PXRD pattern having any 5 peaks selected from the list consisting of: 3.9, 7.8, 9.1, 10.1, 11.6, 14.3, 15.9, 18.2, 20.4, 23.1, 23.9 and 27.0 deg±0.2 degrees 2-theta, a PXRD pattern as depicted in FIG. 12 and combination thereof.

In one embodiment, the present invention encompasses crystalline Sunitinib base characterized by data selected from a group consisting of a PXRD pattern having any 5 peaks selected from the list consisting of: 3.8, 4.2, 8.5, 10.7, 12.7, 13.6, 17.2, 17.7, 26.1 and 27.5 deg±0.2 degrees 2-theta, a PXRD pattern as depicted in FIG. 13 and combination thereof.

In one embodiment, the present invention encompasses crystalline Sunitinib base characterized by data selected from a group consisting of a PXRD pattern having any 5 peaks selected from the list consisting of: 5.3, 8.3, 10.6, 11.0, 11.7, 15.0, 15.7, 16.0 and 27.0 deg±0.2 degrees 2-theta, a PXRD pattern as depicted in FIG. 14.

In one embodiment, the present invention encompasses amorphous Sunitinib base.

In one embodiment, the present invention encompasses crystalline Sunitinib base characterized by data selected from a group consisting of a PXRD pattern having any 5 peaks selected from the list consisting of 8.3, 8.5, 10.5, 13.1, 14.9, 16.0, 18.1, 18.7, 19.2, 20.8, 23.4, 26.1 and 29.5 deg±0.2 degrees 2-theta, a PXRD pattern as depicted in FIG. 16 and combination thereof.

In one embodiment, the present invention encompasses crystalline Sunitinib base characterized by data selected from a group consisting of a PXRD pattern having any 5 peaks selected from the list consisting of: 3.5, 9.3, 9.8, 11.7, 13.9, 14.6, 15.9, 18.1, 21.4 and 26.7 deg±0.2 degrees 2-theta, a PXRD pattern as depicted in FIG. 17 and combination thereof.

In one embodiment, the present invention encompasses crystalline Sunitinib base characterized by data selected from a group consisting of a PXRD pattern having peaks at about 3.9 and 6.0±0.2 degrees 2-theta and any 3 peaks selected from the list consisting of: 3.5, 3.9, 6.0, 9.0, 10.3, 11.8 and 15.0±0.2 degrees 2-theta, a PXRD pattern as depicted in FIG. 18 and combination thereof.

In one embodiment, the present invention encompasses crystalline Sunitinib base characterized by data selected from a group consisting of a PXRD pattern having peaks at about 25.9 and 26.3±0.2 degrees 2-theta and any 3 peaks selected from the list consisting of: 9.0, 10.3, 22.6, 25.9, 26.3 and 27.4±0.2 degrees 2-theta, a PXRD pattern as depicted in FIG. 19 and combination thereof.

In one embodiment, the present invention encompasses crystalline Sunitinib base characterized by data selected from a group consisting of a PXRD pattern having peaks at about 17.2 and 28.3±0.2 degrees 2-theta and any 3 peaks selected from the list consisting of: 4.0, 7.9, 12.0, 17.2, 24.2, 28.3 and 34.9±0.2 degrees 2-theta, a PXRD pattern as depicted in FIG. 20 and combination thereof.

In one embodiment, the present invention encompasses crystalline Sunitinib base characterized by data selected from a group consisting of a PXRD pattern having peaks at about 17.0 and 27.8±0.2 degrees 2-theta and any 3 peaks at positions selected from the group consisting of: 3.9, 10.3, 13.6, 15.8, 17.0, 18.1 and 27.8±0.2 degrees 2-theta, a PXRD pattern as depicted in FIG. 21 and combination thereof.

In one embodiment, the invention encompasses a crystalline form of Sunitinib base characterized by data selected from a group consisting of a PXRD pattern having any 5 peaks at positions selected from the group consisting of: 4.2, 8.5, 10.7, 12.7, 13.6, 17.2, 17.7, 21.1, 26.1 and 27.4±0.2 degrees 2-theta, a PXRD pattern as depicted in FIG. 22 and combination thereof.

In one embodiment, the present invention encompasses crystalline Sunitinib base characterized by data selected from a group consisting of a PXRD pattern having peaks at about 6.6 and 12.6±0.2 degrees 2-theta and any 3 peaks at positions selected from the group consisting of: 15.8, 16.8, 17.4, 23.8 and 26.1±0.2 degrees 2-theta, a PXRD pattern as depicted in FIG. 23 and combination thereof.

In another embodiment, the present invention provides a process for preparing a Sunitinib salt from the above polymorphs of Sunitinib base. Preferably, the sunitinib salt is sunitinib malate. In one embodiment, the process comprises converting the above-described polymorphs of sunitinib base to the Sunitinib salt.

In yet another embodiment, the invention encompasses a process for preparing a sunitinib salt comprising preparing at least one of the above-described polymorphs of sunitinib base according to the process of the present invention, and converting the polymorph to sunitinib salt. Preferably, the sunitinib salt is sunitinib malate.

### BRIEF DESCRIPTION OF THE FIGURES

FIG. 1 shows a powder XRD pattern of crystalline Sunitinib base Form I.

FIG. 2 shows a powder XRD pattern of crystalline Sunitinib base Form II.

FIG. 3 shows a DSC thermogram of crystalline Sunitinib base Form I.

FIG. 4 shows a DSC thermogram of crystalline Sunitinib base Form II.

FIG. 5 shows a powder XRD pattern of crystalline Sunitinib base Form III.

FIG. 6 shows a powder XRD pattern of crystalline Sunitinib base Form IV.

FIG. 7 shows a powder XRD pattern of crystalline Sunitinib base Form V.

FIG. 8 shows a powder XRD pattern of crystalline Sunitinib base Form VI.

FIG. 9 shows a powder XRD pattern of crystalline Sunitinib base Form VII.

FIG. 10 shows a powder XRD pattern of crystalline Sunitinib base Form VIII.

FIG. 11 shows a powder XRD pattern of crystalline Sunitinib base Form IX.

FIG. 12 shows a powder XRD pattern of crystalline Sunitinib base Form X.

FIG. 13 shows a powder XRD pattern of crystalline Sunitinib base Faun XI.

FIG. 14 shows a powder XRD pattern of crystalline Sunitinib base Form XII.

FIG. 15 shows a powder XRD pattern of Amorphous Sunitinib base.

FIG. 16 shows a powder XRD pattern of crystalline Sunitinib base Form XIII.

FIG. 17 shows a powder XRD pattern of crystalline Sunitinib base Form XIV.

FIG. 18 shows a powder XRD pattern of crystalline Sunitinib base Form XV.

FIG. 19 shows a powder XRD pattern of crystalline Sunitinib base Form XVI.

FIG. 20 shows a powder XRD pattern of crystalline Sunitinib base Form XVII.

FIG. 21 shows a powder XRD pattern of crystalline Sunitinib base Form XVIII.

FIG. 22 shows a powder XRD pattern of crystalline Sunitinib base Form D.

FIG. 23: A powder X-ray diffraction pattern of crystalline sunitinib base Form XIX.

### DETAILED DESCRIPTION OF THE INVENTION

As used herein the term “room temperature” refers to a temperature of about 20° C. to about 25° C.

As used herein the term “overnight” refers to a period of about 12 to about 18 hours, preferably for about 14 hours.

The present invention discloses the solid state forms of Sunitinib base and process for their preparation.

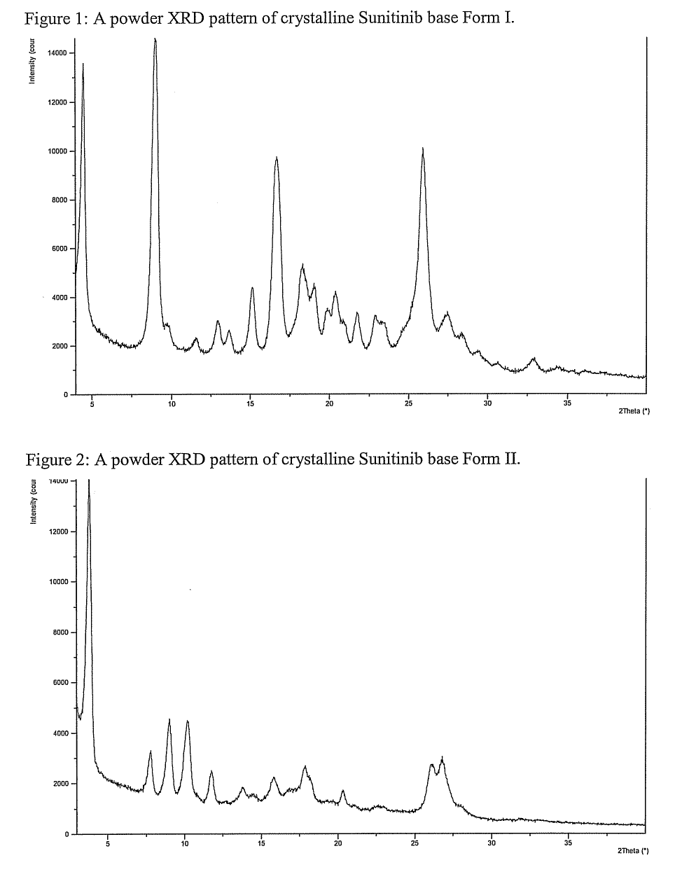

In one embodiment, the present invention encompasses crystalline Sunitinib base characterized by data selected from a group consisting of a PXRD pattern having any 5 peaks selected from the list consisting of: 4.5, 9.0, 15.1, 16.7, 18.3, 19.0, 20.4, 21.8, 25.9 and 27.4 deg±0.2 degrees 2-theta, a PXRD pattern as depicted in FIG. 1 and combination thereof. This form can be designated as “Form I”.

In a preferred embodiment, the present invention encompasses crystalline Sunitinib base designated form I characterized by PXRD pattern having having peaks at about 4.5 and 16.7±0.2 degrees 2-theta and any 3 peaks at positions selected from the group consisting of 9.0, 15.1, 18.3, 19.0, 20.4, 21.8, 25.9 and 27.4 deg±0.2 degrees 2-theta.

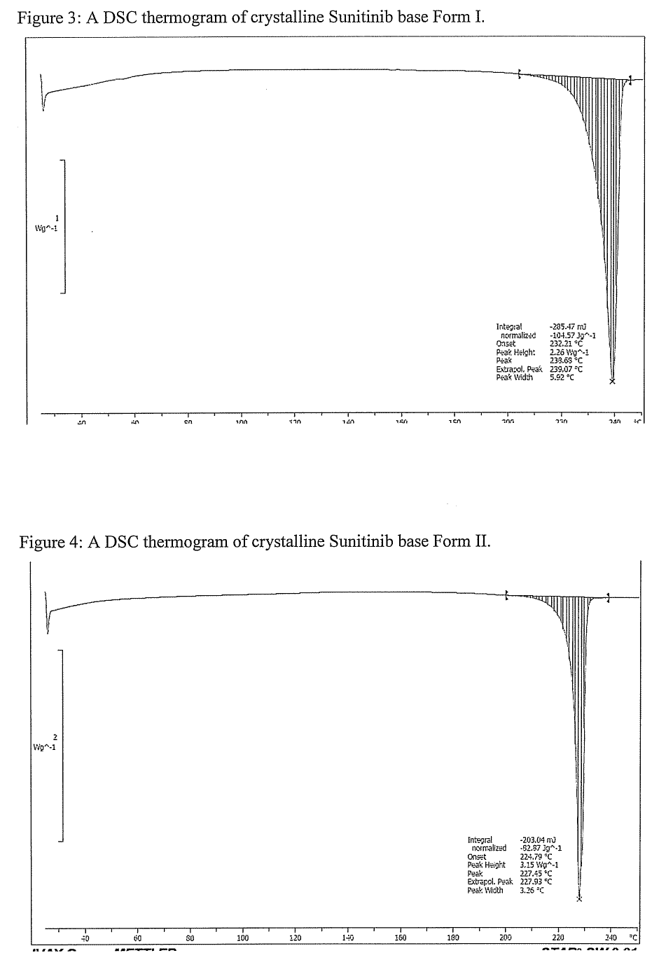

Crystalline Sunitinib base form I can be further characterized by data selected from the group consisting of: a PXRD pattern having peaks at about: 4.5, 15.1, 16.7, 18.3, and 25.9±0.2 degrees 2-theta; a PXRD pattern having peaks at about: 4.5, 9.0, 15.1, 16.7, and 25.9±0.2 degrees 2-theta; a PXRD pattern having peaks at about: 4.5, 16.7, 21.8, 25.9 and 27.4±0.2 degrees 2-theta; a DSC thermogram having an endothermic peak at about 239° C., a DSC thermogram as depicted in FIG. 3; and a weight loss of less than about 1% as measured by TGA. Preferably, this form is anhydrous.

Sunitinib base form I can be prepared by a process comprising precipitating sunitinib base from a mixture comprising 5-Formyl-2,4-dimethyl-1H-pyrrole-3-carboxylic acid, N-(2-diethylaminoethyl)amide, 5-fluoro-1,3-dihydro-indol-2-one, ethanol and triethylamine.

Typically, the precipitation is done by reacting 5-Formyl-2,4-dimethyl-1H-pyrrole-3-carboxylic acid, N-(2-diethylaminoethyl)amide and 5-fluoro-1,3-dihydro-indol-2-one in ethanol and triethylamine providing a suspension comprising the said crystalline sunitinib base. Preferably, 5-Formyl-2,4-dimethyl-1H-pyrrole-3-carboxylic acid, N-(2-diethylaminoethyl) amide and 5-fluoro-1,3-dihydro-indol-2-one in ethanol and triethylamine are combined and the combination is heated. Preferably, the said combination is heated to a temperature of about 50° C. to about 78° C., more preferably, to about 78° C. Preferably, the heating is done for a period of about 2 hours to about 7 hours, more preferably, for about 3 hours.

Typically, the suspension is maintained to increase the yield of the precipitated crystalline form. Preferably, the maintaining is at a temperature of about 30° C. to about 0° C., preferably, to about 25° C. to about 20° C. Preferably, maintaining is done for overnight. More preferably, maintaining is done for about 10 hours to 18 hours.

The process for preparing the said crystalline Sunitinib base form I may further comprise recovering the crystalline sunitinib base from the cooled suspension. The recovery may be done for example by filtering the suspension, washing the filtered precipitate of the crystalline form and drying. Preferably, drying is done at a temperature of about 40° C. to about 90° C., more preferably, at a temperature of about 50° C. to about 80° C., most preferably, at about 70° C. Preferably, drying is done for about 3 hours to about 16 hours, more preferably, for about 4 hours.

In one embodiment, the present invention encompasses crystalline Sunitinib base characterized by data selected from a group consisting of a PXRD pattern having any 5 peaks selected from the list consisting of: 3.8, 7.8, 9.0, 10.2, 11.8, 15.8, 17.9, 20.3, 26.1 and 26.8 deg±0.2 degrees 2-theta, a PXRD pattern as depicted in FIG. 2 and combination thereof. This form can be designated as “Form II”.

In a preferred embodiment, the present invention encompasses crystalline Sunitinib base designated form II characterized by PXRD pattern having peaks at about 9.0 and 26.1±0.2 degrees 2-theta and any 3 peaks at positions selected from the group consisting of 3.8, 7.8, 10.2, 11.8, 15.8, 17.9, 20.3 and 26.8 deg±0.2 degrees 2-theta.

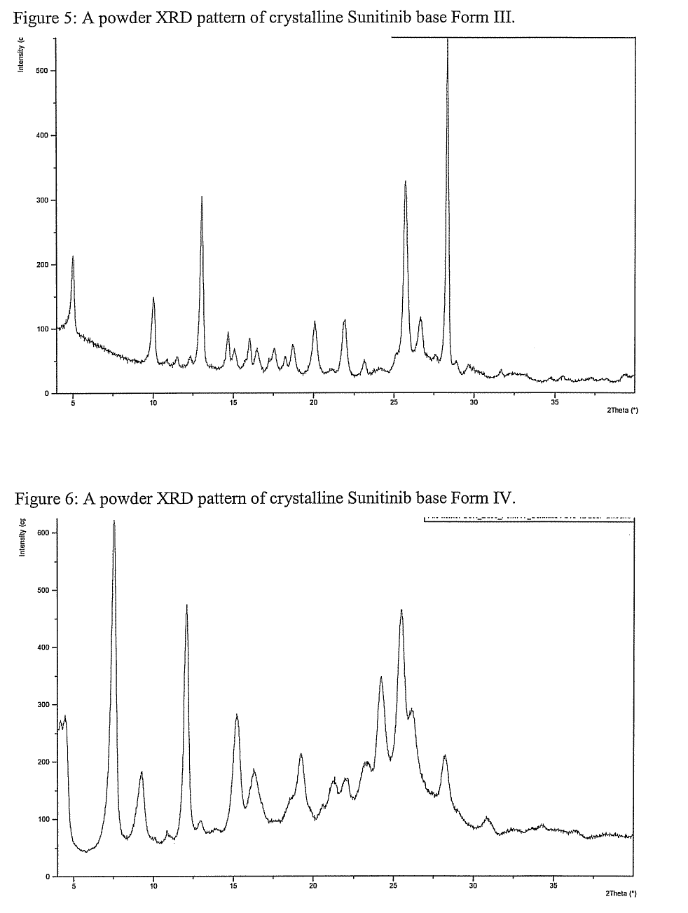

Crystalline Sunitinib base form II can be further characterized by data selected from the group consisting of: a PXRD pattern having peaks at about: 7.8, 9.0, 10.2, 11.8, 26.1 and 26.8±0.2 degrees 2-theta; a PXRD pattern having peaks at about: 7.8, 10.2, 11.8, 17.9, and 26.1±0.2 degrees 2-theta; a PXRD pattern having peaks at about: 3.8, 7.8, 10.2, 11.8, and 15.8±0.2 degrees 2-theta; a DSC thermogram having an endothermic peak at about 228° C., a DSC thermogram as depicted in FIG. 4; and a weight loss of less than about 1% as measured by TGA.

Sunitinib base form II can be prepared by a process comprising dissolving Sunitinib base in acidic water, and precipitating at a temperature of about room temperature by adding base, thus providing a suspension comprising the said crystalline form, wherein the ratio of Sunitinib base to water is about 1:6 (w:w).

Preferably, the said solution is provided by providing a mixture of sunitinib base and water, combining the said mixture with an inorganic acid and heating the said combination.

Preferably, the acid is added to the said mixture. Preferably, the acid is a mineral acid. Preferably, the mineral acid is hydrochloric acid. Preferably, the addition of the acid provides sunitinib salt. Preferably, the addition of the acid provides a pH of about 1.0 to about 3.0, more preferably of about 1.5.

Preferably, the dissolution step comprises heating the mixture to a temperature of about 30° C. to about 70° C., more preferably, to a temperature of about 40° C. to about 60° C., most preferably, to a temperature of about 40° C.

Preferably, the base, which is used in the precipitation step, is an organic or inorganic base, more preferably, ammonium hydroxide or sodium hydroxide, most preferably, ammonium hydroxide.

Preferably, ammonium hydroxide is added providing a pH of about 8 to about 10, more preferably, a pH of about 8.5. Preferably, the ammonium hydroxide is added is in a form of an aqueous solution. Preferably, the aqueous solution has a concentration of about 25% w/w.

Optionally, the process may further comprise purifying the said solution prior to the precipitation of the crystalline form. The purification comprises extracting the said solution with methyl isobutyl ketone, and optionally also with dimethylacetamide.

The process for preparing the said crystalline Sunitinib base form II may further comprise recovering the crystalline sunitinib base from the suspension. The recovery may be done for example by filtering the suspension, washing the filtered precipitate of the crystalline form and drying; preferably, drying is done at a temperature of about 40° C. to about 90° C., more preferably, at a temperature of about 50° C. to about 80° C., most preferably, at about 70° C. Preferably, drying is done for overnight.

In one embodiment, the present invention encompasses crystalline Sunitinib base characterized by data selected from a group consisting of a PXRD pattern having any 5 peaks selected from the list consisting of: 5.0, 10.0, 13.0, 14.7, 16.0, 20.1, 21.9, 25.7, 26.6 and 28.3 deg±0.2 degrees 2-theta, a PXRD pattern as depicted in FIG. 5 and combination thereof. This form can be designated as “Faun III”.

In a preferred embodiment, the present invention encompasses crystalline Sunitinib base designated form III characterized by PXRD pattern having peaks at about 5.0 and 13.0±0.2 degrees 2-theta and any 3 peaks at positions selected from the group consisting of 10.0, 14.7, 16.0, 20.1, 21.9, 25.7, 26.6 and 28.3 deg±0.2 degrees 2-theta.

Crystalline Sunitinib base form III can be further characterized by data selected from the group consisting of: a PXRD pattern having peaks at about: 5.0, 10.0, 13.0, 25.7 and 28.3±0.2 degrees 2-theta; a PXRD pattern having peaks at about: 5.0, 13.0, 14.7, 20.1 and 28.3±0.2 degrees 2-theta; a PXRD pattern having peaks at about: 5.0, 13.0, 20.1, 21.9 and 28.3±0.2 degrees 2-theta; and a water content of up to 1.5% by weight as measured by KF.

Sunitinib base form III can be prepared by a process comprising crystallizing sunitinib from a mixture comprising a first polar aprotic organic solvent and acetone, wherein the first polar aprotic organic solvent is selected from the group consisting of 1-methyl-2-pyrrolidone (“NMP”), dimethylacetamide (“DMA”), dimethylformamide (“DMF”) or dimethylsulfoxide (“DMSO”), and mixtures thereof.

Typically, the crystallization comprises providing a solution of sunitinib base and a first polar aprotic organic solvent selected from the group consisting of NMP, DMA, DMF or DMSO, and mixtures thereof, and combining the solution with acetone to obtain a suspension comprising the said crystalline form III.

Preferably, the first polar aprotic organic solvent is either NMP or DMA.

Preferably, the said solution is provided by combining sunitinib base and the first polar aprotic organic solvent and heating the combination. Preferably the heating is done to a temperature of about 60° C. to about 100° C., more preferably of about 85° C. to about 90° C., most preferably at about 90° C.

Preferably, acetone is added to the solution.

Typically, to increase the yield of the precipitated crystalline sunitinib base, precipitation can be followed by cooling the said suspension to a temperature of about 30° C. to about 0° C., more preferably, to a temperature of about 25° C. to about 20° C. Preferably, the cooling is conducted for overnight.

The process for preparing the said crystalline Sunitinib base form III may further comprise recovering the crystalline sunitinib base from the suspension. The recovery may be done for example, by filtering the suspension, and drying. Preferably, the drying is done at a temperature of about 40° C. to about 90° C., more preferably, at a temperature of about 50° C. to about 80° C., most preferably, at about 50° C. Preferably, the drying is performed for a period of 4 hours.

In one embodiment, the present invention encompasses crystalline Sunitinib base characterized by data selected from a group consisting of a PXRD pattern having any 5 peaks selected from the list consisting of: 7.6, 9.3, 12.1, 15.2, 16.3, 19.2, 24.2, 25.5, 26.2 and 28.2 deg±0.2 degrees 2-theta, a PXRD pattern as depicted in FIG. 6. This form can be designated as “Form IV”.

In a preferred embodiment, the present invention encompasses crystalline Sunitinib base designated form IV characterized by PXRD pattern peaks at about 7.6 and 15.2±0.2 degrees 2-theta and any 3 peaks at positions selected from the group consisting of 9.3, 12.1, 16.3, 19.2, 24.2, 25.5, 26.2 and 28.2 deg±0.2 degrees 2-theta.

Crystalline Sunitinib base form IV can be further characterized by data selected from the group consisting of: a PXRD pattern having peaks at about: 7.6, 12.1, 15.2, 24.2 and 25.5±0.2 degrees 2-theta; a PXRD pattern having peaks at about: 7.6, 9.3, 15.2, 19.2 and 24.2±0.2 degrees 2-theta; and a PXRD pattern having peaks at about: 7.6, 12.1, 24.2, 25.5 and 28.2±0.2 degrees 2-theta.

The above crystalline of sunitinib base is prepared by a process comprising lyophilizing a solution of sunitinib base in t-butanol. Lyophilization is also known as freeze drying.

In the lyophilization process, a solution of sunitinib base in t-butanol is frozen, and then the frozen mass is subjected to a pressure of less than about one atmosphere, to remove the solvent.

Preferably, the solution is provided by a process comprising combining sunitinib base and t-butanol and heating the combination. Preferably, the heating is to a temperature of about 80° C. Preferably, freezing the solution is done gradually. First, cooling to a temperature of about 25° C. is done, and then cooling to a temperature of about −20° C. is performed, providing a frozen solution.

Typically, the evaporation of the solvents is done at about −20° C. Preferably, evaporation of the solvent is done under reduced pressure (less than one atmosphere). Preferably, the reduced pressure is used in the range 1 mBar.

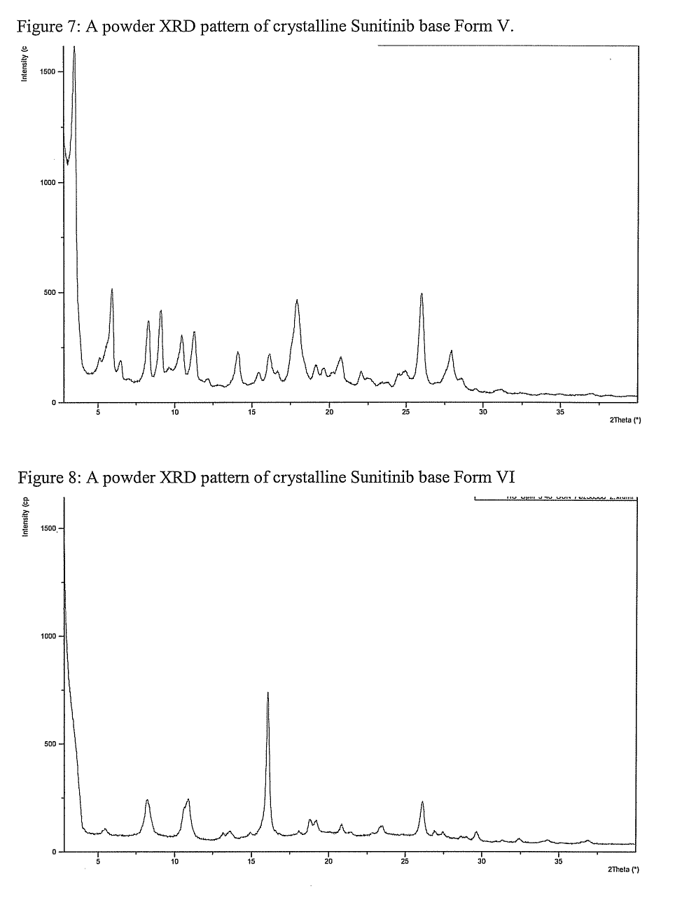

In one embodiment, the present invention encompasses crystalline Sunitinib base characterized by data selected from a group consisting of a PXRD pattern having any 5 peaks selected from the list consisting of: 3.5, 5.9, 6.5, 8.3, 10.5, 11.3, 14.1, 17.9, 20.7, 26.0 and 27.9 deg±0.2 degrees 2-theta, a PXRD pattern as depicted in FIG. 7 and combination thereof.

In one embodiment, the present invention encompasses crystalline Sunitinib base characterized by data selected from a group consisting of a PXRD pattern having any 5 peaks selected from the list consisting of: 3.5, 5.9, 6.5, 8.3, 10.5, 11.3, 14.1, 17.9, 20.7, 26.0 and 27.9 deg±0.2 degrees 2-theta, a PXRD pattern as depicted in FIG. 7 and combination thereof. This fouu can be designated as “Form V”.

In a preferred embodiment, the present invention encompasses crystalline Sunitinib base designated Ruin V characterized by PXRD pattern having peaks at about 5.9 and 11.3±0.2 degrees 2-theta and any 3 peaks at positions selected from the group consisting of 3.5, 6.5, 8.3, 10.5, 14.1, 17.9 and 27.9 deg±0.2 degrees 2-theta.

Crystalline Sunitinib base form V can be further characterized by data selected from the group consisting of: a PXRD pattern having peaks at about: 3.5, 5.9, 8.3, 14.1 and 27.9±0.2 degrees 2-theta; a PXRD pattern having peaks at about: 5.9, 8.3, 14.1, 17.9, and 26.0±0.2 degrees 2-theta; and a PXRD pattern having peaks at about: 3.5, 5.9, 11.3, 17.9 and 27.9±0.2 degrees 2-theta.

Sunitinib base form V can be prepared by a process comprising crystallizing sunitinib base from pyridine.

The crystallization comprises providing a solution of sunitinib base and pyridine, and precipitating the said crystalline form to obtain a suspension.

Preferably, the said solution is provided by combining sunitinib base and pyridine and heating the combination. Preferably the heating is done to a temperature of about 115° C.

Preferably, the precipitation is done by cooling and maintaining the said solution. Preferably, cooling the said solution is done to a temperature of about 25° C. Preferably, the maintaining is conducted for about 5 hours.

The process for preparing the said crystalline Sunitinib base form V may further comprise recovering the crystalline sunitinib base from the suspension. The recovery may be done for example, by filtering the suspension, washing the filtered precipitate and drying. Preferably, the drying can be done at about room temperature in air. Preferably, the washing is done with tert-butylmethyl ether.

In one embodiment, the present invention encompasses crystalline Sunitinib base characterized by data selected from a group consisting of a PXRD pattern having any 5 peaks selected from the list consisting of: 8.2, 10.9, 13.2, 13.6, 16.1, 18.8, 19.2, 20.9, 23.5, 26.1 and 29.6 deg±0.2 degrees 2-theta, a PXRD pattern as depicted in FIG. 8 and combination thereof. This form can be designated as form VI.

In a preferred embodiment, the present invention encompasses crystalline Sunitinib base designated form VI characterized by PXRD pattern having peaks at about 10.9 and 16.1±0.2 degrees 2-theta and any 3 peaks at positions selected from the group consisting of 8.2, 13.6, 18.8, 19.2, 26.1 and 29.6 deg±0.2 degrees 2-theta.

In another embodiment, the present invention encompasses crystalline Sunitinib base characterized by PXRD pattern as depicted in FIG. 8. This form can be designated form VI.

Crystalline Sunitinib base form VI can be further characterized by data selected from the group consisting of: a PXRD pattern having peaks at about: 8.2, 10.9, 16.1, 23.5 and 29.6±0.2 degrees 2-theta; a PXRD pattern having peaks at about: 5.5, 10.9, 16.1, 20.9 and 26.1±0.2 degrees 2-theta; and a PXRD pattern having double peak at about 18.7 and 19.2±0.2 degrees 2-theta.

Sunitinib base form VI can be prepared by a process comprising crystallizing sunitinib base from a mixture of tetrahydrofuran and water

The crystallization comprises providing a solution of sunitinib base in a mixture of tetrahydrofuran and water, and precipitating the said crystalline form to obtain a suspension. Preferably, the said solution is provided by combining sunitinib base with a mixture of tetrahydrofuran and water, and heating the combination. Preferably, the precipitation is done by cooling the said solution, and partial evaporating the solvent to obtain a precipitation in a form of a gel. Preferably, cooling is done to about room temperature. Preferably, evaporating the said solution is done for about overnight.

The process for preparing the said crystalline Sunitinib base form VI may further comprise recovering the crystalline sunitinib base. The recovery may be done for example, by drying. Preferably, the drying is done at a temperature of about 50° C., preferably at a pressure of about 1 mBar, preferably, drying is conducted for 2 hours

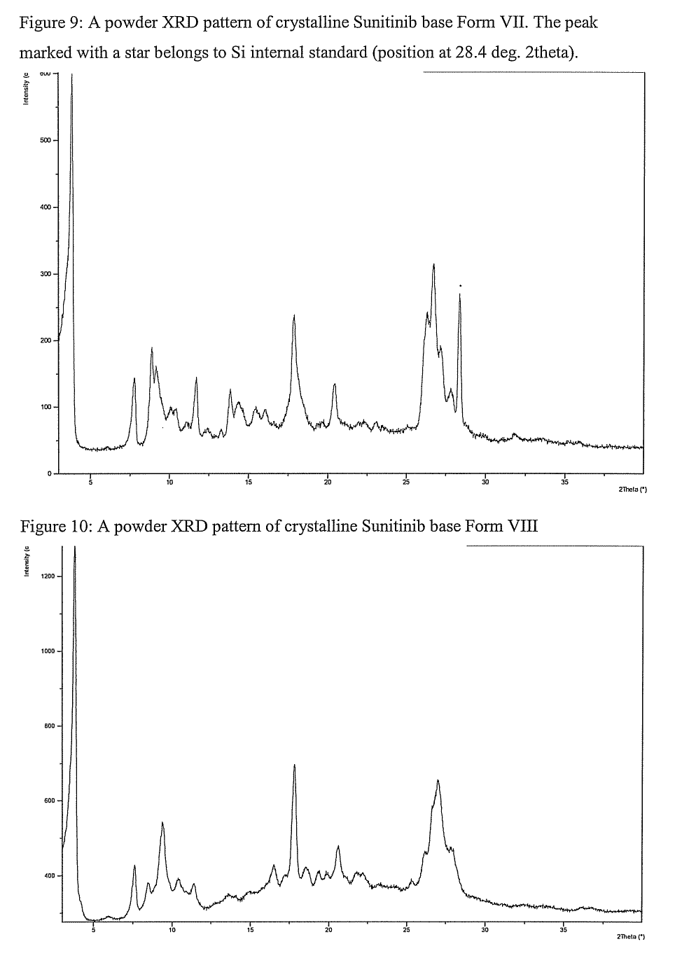

In one embodiment, the present invention encompasses crystalline Sunitinib base characterized by data selected from a group consisting of a PXRD pattern having any 5 peaks selected from the list consisting of: 3.9, 7.8, 8.9, 9.2, 11.7, 13.9, 15.4, 16.0, 17.9, 20.4, 26.7 and 27.8 deg±0.2 degrees 2-theta, a PXRD pattern as depicted in FIG. 9 and combination thereof. This form can be designated form VII.

In a preferred embodiment, the present invention encompasses crystalline Sunitinib base designated form VII characterized by PXRD pattern having peaks at about 8.9 and 9.2±0.2 degrees 2-theta and any 3 peaks at positions selected from the group consisting of 3.9, 7.8, 11.7, 13.9, 17.9, 20.4 and 26.7 deg±0.2 degrees 2-theta.

The peak marked with a star belongs to Si internal standard (position at 28.4 deg.±0.2 degrees 2theta).

Crystalline Sunitinib base form VII can be further characterized by data selected from the group consisting of: a PXRD pattern having double peak at about: 3.9, 7.8, 8.9, 9.2 and 11.7±0.2 degrees 2-theta; a PXRD pattern having double peak at about: 8.9 and 9.2±0.2 degrees 2-theta and a PXRD pattern having double peak at about 15.4 and 16.0±0.2 degrees 2-theta.

Sunitinib base form VII can be prepared by a process comprising a) providing a mixture of Sunitinib base in tetrahydrofuran, b) recovering Sunitinib base from the mixture, c) dissolving Sunitinib base in acidic water, and d) precipitating the said crystalline form by adding a base to the solution in step c.

Preferably, the mixture of Sunitinib base in tetrahydrofuran is provided by reacting Sunitinib activated carboxylic acid derivative with N,N-diethylaminoethylamine in tetrahydrofuran.

The starting Sunitinib base can be prepared for example, according to the process in example 24.

Preferably, step b is done by evaporation of the solvent.

Preferably, step c is done by combining sunitinib base and a mixture of water and hydrochloric acid and heating the said combination to obtain a solution. The above solution is then optionally washed, preferably, with methyl isobutyl ketone. Preferably, washing is done at a temperature of about 50° C. to about 60° C., more preferably, of about 50° C.

Preferably, the base, which is used in step d, is an organic or an inorganic base, more preferably, ammonium hydroxide or sodium hydroxide, most preferably, ammonium hydroxide. Preferably, the addition of the base in step d provides a pH of about 8.5 to about 9.5, more preferably, of about 9.

The process for preparing the said crystalline Sunitinib base may further comprise recovering the crystalline sunitinib base. The recovery may be done for example, by filtering and drying. Preferably, the drying is done at a temperature of about 55° C. to about 65° C., more preferably, of about 60° C. Preferably, drying is conducted for a period of about 10 hours to about 18 hours, more preferably, of about 16 hours.

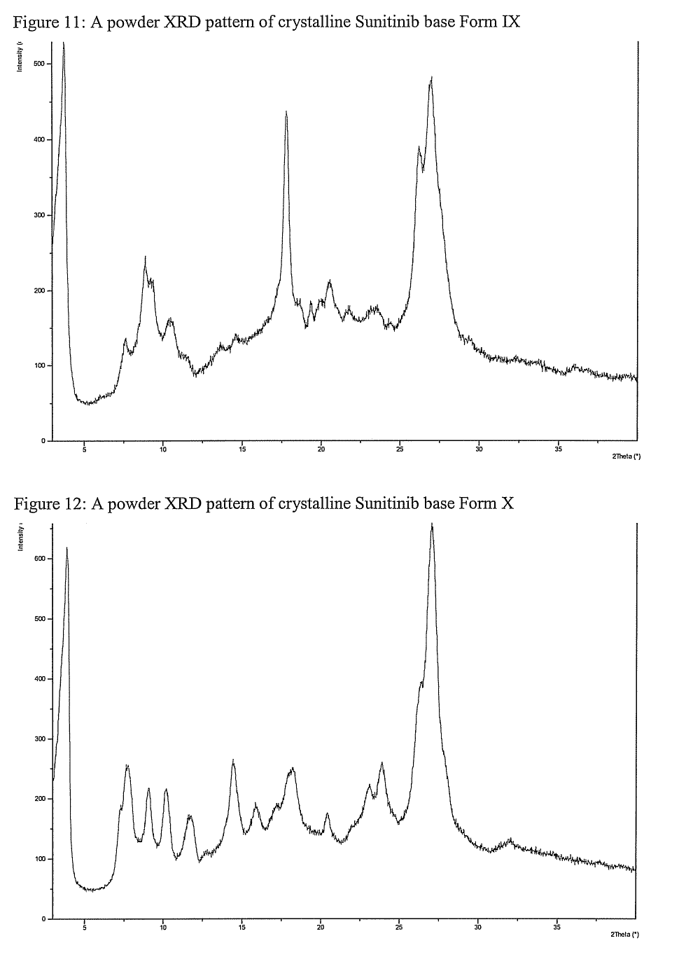

In one embodiment, the present invention encompasses crystalline Sunitinib base characterized by data selected from a group consisting of a PXRD pattern having any 5 peaks selected from the list consisting of: 3.8, 7.6, 8.5, 9.5, 10.4, 11.4, 16.5, 17.8, 20.6, and 27.0 deg±0.2 degrees 2-theta, a PXRD pattern as depicted in FIG. 10 and combination thereof. This form can be designated form VIII.

In a preferred embodiment, the present invention encompasses crystalline Sunitinib base designated form VIII characterized by PXRD pattern having peaks at about 7.6 and 16.5±0.2 degrees 2-theta and any 3 peaks at positions selected from the group consisting of 3.8, 8.5, 9.5, 11.4, 17.8, 20.6 and 27.0 deg±0.2 degrees 2-theta.

Crystalline Sunitinib base form VIII can be further characterized by data selected from the group consisting of: a PXRD pattern having peaks at about: 3.8, 7.6, 9.5, 16.5 and 20.6±0.2 degrees 2-theta; a PXRD pattern having peaks at about: 3.8, 7.6, 9.5, 17.8 and 27.0±0.2 degrees 2-theta; and a PXRD pattern having peaks at about 3.8, 7.6, 9.5, 10.4 and 11.4±0.2 degrees 2-theta.

Sunitinib base form VIII can be prepared by a process comprising a) providing a mixture of Sunitinib base and 2-methyltetrahydrofuran, b) combining the mixture of step a with acidic water to obtain a solution, c) cooling the said solution and d) precipitating the said crystalline form by adding base to the cooled solution of step c. Preferably, the mixture of Sunitinib base in 2-methyltetrahydrofuran is provided by reacting Sunitinib activated carboxylic acid derivative with N,N-diethylaminoethylamine in 2-methyltetrahydrofuran.

The starting Sunitinib base can be prepared for example, according to the process in example 25.

Preferably, the dissolution is achieved at a temperature of about 70° C.

Preferably, the cooling step is done to a temperature of about 50° C. to about 25° C.

Preferably, the base, which is used in step e, is an organic or an inorganic base, more preferably, ammonium hydroxide or sodium hydroxide, most preferably, ammonium hydroxide. Preferably, the addition of the base in step d provides a pH of about 8.5.

The process for preparing the said crystalline Sunitinib base may further comprise recovering the crystalline sunitinib base. The recovery may be done for example, by filtering and drying. Preferably, the drying is done at a temperature of about 55° C. to about 65° C., more preferably, of about 60° C. Preferably, drying is conducted for a period of about 10 hours to about 18 hours, more preferably, of about 16 hours.

In one embodiment, the present invention encompasses crystalline Sunitinib base characterized by data selected from a group consisting of a PXRD pattern having any 5 peaks selected from the list consisting of: 3.8, 7.6, 8.9, 9.3, 10.4, 17.8, 20.5, 21.7, 26.2, and 26.9 deg±0.2 degrees 2-theta, a PXRD pattern as depicted in FIG. 11 and combination thereof. This form can be designated form IX.

In a preferred embodiment, the present invention encompasses crystalline Sunitinib base designated form IX characterized by PXRD pattern peaks at about 8.9 and 26.9±0.2 degrees 2-theta and any 3 peaks at positions selected from the group consisting of 3.8, 7.6, 10.4, 17.8, 20.5 and 26.2 deg±0.2 degrees 2-theta.

Crystalline Sunitinib base form IX can be further characterized by data selected from the group consisting of: a PXRD pattern having peaks at about: 3.8, 8.9, 17.8, 20.5 and 26.2 and ±0.2 degrees 2-theta; a PXRD pattern having peaks at about: 3.8, 8.9, 17.8, 26.2 and 26.91 0.2 degrees 2-theta; and a PXRD pattern having peaks at about: 3.8, 7.6, 8.9, 13.4 and 17.8±0.2 degrees 2-theta.Sunitinib base form IX can be prepared by a process comprising a) providing a mixture of Sunitinib base and 2-methyltetrahydrofuran b) combining the mixture of step a with acidic water to obtain a solution, c) precipitating the said crystalline form by adding base to the solution in step b to obtain a suspension and d) heating the said suspension in step c to obtain the said crystalline form.

Preferably, the mixture of Sunitinib base in 2-methyltetrahydrofuran is provided by reacting Sunitinib activated carboxylic acid derivative with N,N-diethylaminoethylamine in 2-methyltetrahydrofuran.

The starting Sunitinib base can be prepared for example, according to the process in example 25.

Preferably, the dissolution step is done at a temperature of about 70° C.

Preferably, the heating is done to a temperature of about 50° C.

Preferably, the base, which is used in step d, is an organic or an inorganic base, more preferably, ammonium hydroxide or sodium hydroxide, most preferably, ammonium hydroxide. Preferably, the addition of the base in step d provides a pH of about 8.5.

The process for preparing the said crystalline Sunitinib base may further comprise recovering the crystalline sunitinib base. The recovery may be done for example, by filtering and drying. Preferably, the filtering is done at a temperature of about 50° C. Preferably, the drying is done at a temperature a temperature of about 55° C. to about 65° C., more preferably, of about 60° C. Preferably, drying is conducted for a period of about 10 hours to about 18 hours, more preferably, of about 16 hours.

In one embodiment, the present invention encompasses crystalline Sunitinib base characterized by data selected from a group consisting of a PXRD pattern having any 5 peaks selected from the list consisting of: 3.9, 7.8, 9.1, 10.1, 11.6, 14.3, 15.9, 18.2, 20.4, 23.1, 23.9 and 27.0 deg±0.2 degrees 2-theta, a PXRD pattern as depicted in FIG. 12 and combination thereof. This form can be designated form X.

In a preferred embodiment, the present invention encompasses crystalline Sunitinib base designated form X characterized by PXRD pattern having peaks at about 14.3 and 23.9±0.2 degrees 2-theta and any 3 peaks at positions selected from the group consisting of 3.9, 7.8, 9.1, 10.1, 11.6, 23.1 and 27.0 deg±0.2 degrees 2-theta.

Crystalline Sunitinib base form X can be further characterized by data selected from the group consisting of a PXRD pattern having peaks at about: 3.9, 7.8, 9.1, 10.1 and 11.6; a PXRD pattern having peaks at about: 3.9, 7.8, 14.3, 23.9 and 23.0 and ±0.2 degrees 2-theta; and a PXRD pattern having peaks at about: 3.9, 7.8, 18.2, 23.1 and 23.9±0.2 degrees 2-theta.

Sunitinib base form X can be prepared by a process comprising dissolving Sunitinib base in acidic water, and precipitating by adding a base thus providing a suspension comprising the said crystalline form, wherein the ratio of Sunitinib base to water is about 1:28.5(w:w).

Preferably, the dissolution step is done at a temperature of about 50° C. to about 60° C. The above solution is then washed, preferably, with methyl isobutyl ketone.

Preferably, the base, which is used in the precipitation step, is an organic or inorganic base, more preferably, ammonium hydroxide or sodium hydroxide, most preferably, ammonium hydroxide.

Preferably, ammonium hydroxide is added providing a pH of about 8 to about 10, more preferably, a pH of about 9. Preferably, the ammonium hydroxide is added in a form of an aqueous solution. Preferably, the aqueous solution has a concentration of about 30% w/w.

The process for preparing the said crystalline Sunitinib base may further comprise recovering the crystalline sunitinib base. The recovery may be done for example, by filtering and drying. Preferably, the drying is done at a temperature of about 55° C. to about 65° C., more preferably, of about 60° C.

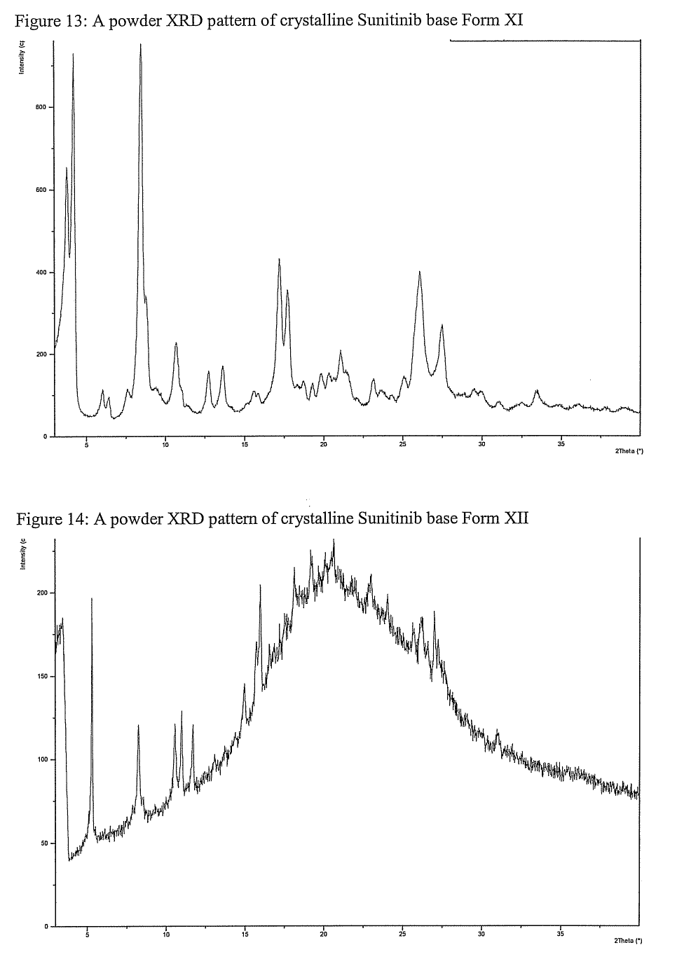

In one embodiment, the present invention encompasses crystalline Sunitinib base characterized by data selected from a group consisting of a PXRD pattern having any 5 peaks selected from the list consisting of: 3.8, 4.2, 8.5, 10.7, 12.7, 13.6, 17.2, 17.7, 26.1 and 27.5 deg±0.2 degrees 2-theta, a PXRD pattern as depicted in FIG. 13 and combination thereof. This form can be designated form XI.

In a preferred embodiment, the present invention encompasses crystalline Sunitinib base designated form XI characterized by PXRD pattern having peaks at about 4.2 and 6.0±0.2 degrees 2-theta and any 3 peaks at positions selected from the group consisting of 3.8, 8.5, 10.7, 12.7, 13.6, 17.2 and 27.5 deg±0.2 degrees 2-theta.

Crystalline Sunitinib base form XI can be further characterized by data selected from the group consisting of: a PXRD pattern having peaks at about: 4.2, 8.5, 12.7, 13.6 and 17.2; a PXRD pattern having double peak at about: 3.8 and 4.2 and ±0.2 degrees 2-theta; and a PXRD pattern having double peak at about: 17.2 and 17.7±0.2 degrees 2-theta.

Sunitinib base form XI can be prepared by a process comprising crystallizing sunitinib base from toluene.

The crystallization comprises combining Sunitinib base and toluene to obtain a solution, and precipitation the said crystalline form to obtain a suspension.

Preferably, the said solution is provided by heating the said combination. Preferably the heating is done to a temperature of about 101° C.

Preferably, the precipitation is done by cooling the said solution. Preferably, cooling the said solution is done to a temperature of about 20° C. Preferably, the cooled mixture is conducted maintained is conducted for about 3 hours.

The process for preparing the said crystalline sunitinib base form XI may further comprises recovering the crystalline Sunitinib base from the suspension. The recovery may be done for example, by filtering and drying the suspension. The drying can be done at about room temperature.

In one embodiment, the present invention encompasses crystalline Sunitinib base characterized by data selected from a group consisting of a PXRD pattern having any 5 peaks selected from the list consisting of: 5.3, 8.3, 10.6, 11.0, 11.7, 15.0, 15.7, 16.0 and 27.0 deg±0.2 degrees 2-theta, a PXRD pattern as depicted in FIG. 14 and combination thereof. This form can be designated form XII

In a preferred embodiment, the present invention encompasses crystalline Sunitinib base designated form XII characterized by PXRD pattern having peaks at about 5.3 and 8.3±0.2 degrees 2-theta and any 3 peaks at positions selected from the group consisting of 10.6, 11.0, 11.7, 15.0, 15.7, 16.0 and 27.0 deg±0.2 degrees 2-theta.

Crystalline Sunitinib base form XII can be further characterized by data selected from the group consisting of: a PXRD pattern having peaks at about: 5.3, 8.3, 11.0, 11.7 and 15.7; and a PXRD pattern having double peak at about: 10.6 and 11.0±0.2 degrees 2-theta.

Sunitinib base form XII can be prepared by a process comprising providing a mixture of Sunitinib base and tetrahydrofuran to obtain a solution, and evaporating the said solvent to obtain the said crystalline, wherein the ratio of Sunitinib base to tetrahydrofuran is about 1:33 (g/ml).

Preferably, the said solution is provided by heating the said mixture. Preferably the heating is done to a temperature of about 66° C. Preferably, evaporating the said solvent is conducted at a temperature of about 20° C. Preferably the evaporating is conducted on air. Preferably, the evaporating of the said solvent is conducted till drying to get the said crystalline form.

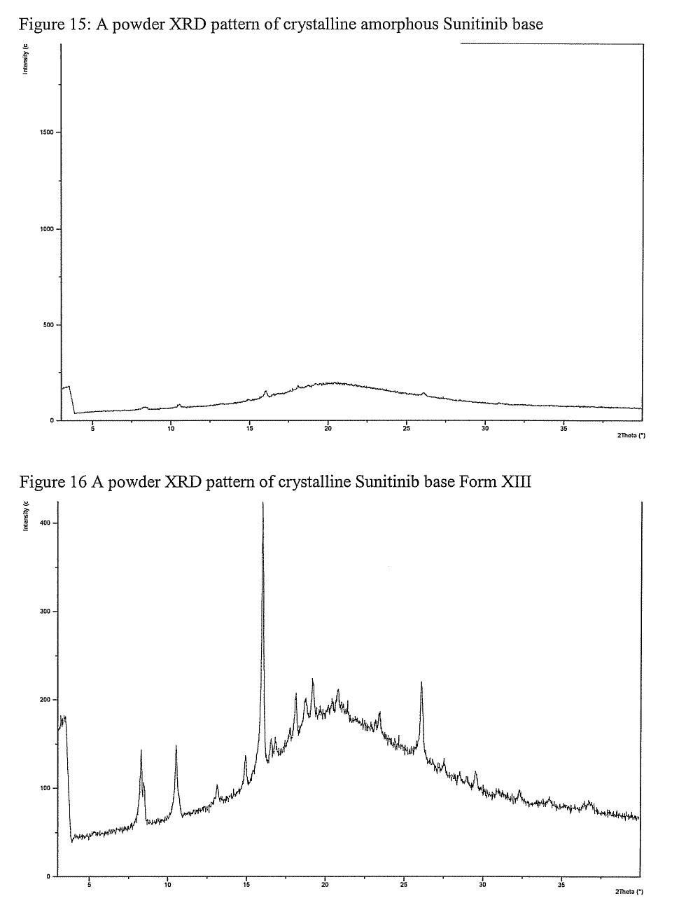

In one embodiment, the present invention encompasses amorphous Sunitinib base. The amorphous sunitinib base can be characterized by PXRD pattern having broad diffraction peak, as depicted in FIG. 15.

In another embodiment, the invention encompasses a process for preparing amorphous sunitinib base comprising providing a mixture of Sunitinib base and tetrahydrofuran to obtain a solution, and evaporating the said solvent.

Preferably, the said solution is provided by heating the said mixture. Preferably the heating is done to a temperature of about 66° C. Preferably, evaporating the said solvent is conducted at a temperature of about 20° C.

Preferably, the evaporating of the said solvent is conducted till drying to obtain the amorphous sunitinib base. Preferably the evaporating is conducted in air.

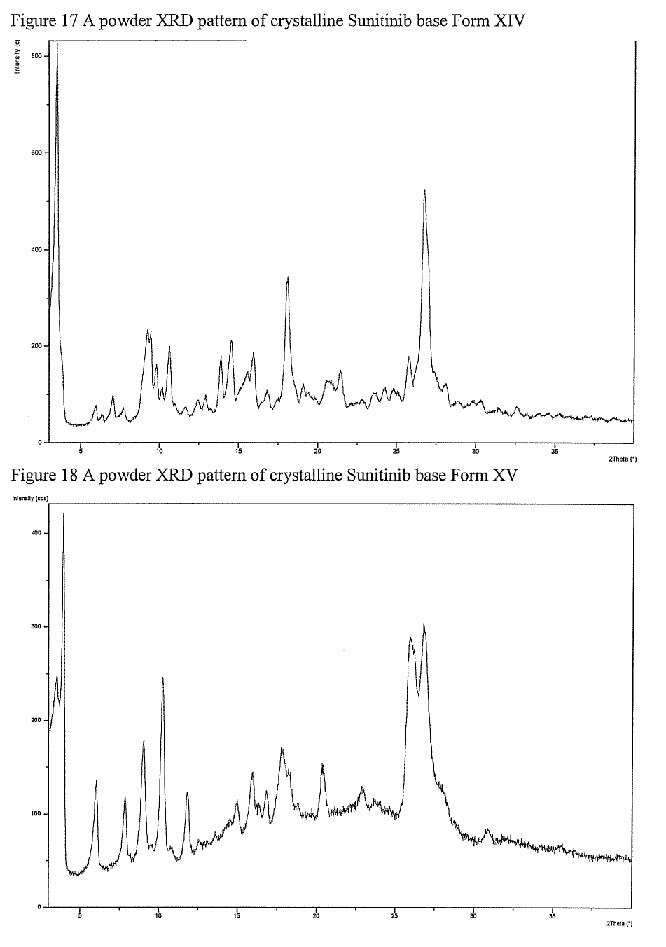

In one embodiment, the present invention encompasses crystalline Sunitinib base characterized by data selected from a group consisting of a PXRD pattern having any 5 peaks selected from the list consisting of: 8.3, 8.5, 10.5, 13.1, 14.9, 16.0, 18.1, 18.7, 19.2, 20.8, 23.4, 26.1 and 29.5 deg±0.2 degrees 2-theta, a PXRD pattern as depicted in FIG. 16 and combination thereof. This limn can be designated form XIII.

In a preferred embodiment, the present invention encompasses crystalline Sunitinib base desidnated form XIII characterized by PXRD pattern having peaks at about 8.3 and 16.0±0.2 degrees 2-theta and any 3 peaks at positions selected from the group consisting of 10.5, 14.9, 18.1, 23.4, 26.1 and 29.5 deg±0.2 degrees 2-theta.

Crystalline Sunitinib base form XIII can be further characterized by data selected from the group consisting of: a PXRD pattern having peaks at about: 13.1, 18.7, 23.4, 26.1 and 29.5; a PXRD pattern having peaks at about: 18.7, 23.4 and 29.5; and a PXRD pattern having double peak at about: 8.3 and 8.5±0.2 degrees 2-theta.Sunitinib base form XIII can be prepared by a process comprising crystallizing sunitinib base from a mixture of tetrahydrofuran and water.

The crystallization comprises providing a solution of Sunitinib base in a mixture of tetrahydrofuran and water, and evaporating the said solvent to obtain the said crystalline. Preferably, the said solution is provided by combining sunitinib base, tetrahydrofuran and water, and heating the said combination. Preferably the heating is done to a temperature of about 66° C.

Preferably, the heating is conducted for a period of about 10 min.

Preferably, evaporating the said solvent is conducted at a temperature of about 20° C.

Preferably, the evaporating the said solvent is conducted till drying to get the said crystalline form. Preferably the evaporating is conducted in air.

In one embodiment, the present invention encompasses crystalline Sunitinib base characterized by data selected from a group consisting of a PXRD pattern having any 5 peaks selected from the list consisting of: 3.5, 9.3, 9.8, 11.7, 13.9, 14.6, 15.9, 18.1, 21.4 and 26.7 deg±0.2 degrees 2-theta, a PXRD pattern as depicted in FIG. 17 and combination thereof. This form can be designated form XIV.

In one embodiment, the present invention encompasses crystalline Sunitinib base characterized by data selected from a group consisting of a PXRD pattern having any 5 peaks selected from the list consisting of: 3.5, 9.3, 9.8, 11.7, 13.9, 14.6, 15.9, 18.1, 21.4 and 26.7 deg±0.2 degrees 2-theta, a PXRD pattern as depicted in FIG. 17 and combination thereof.

In a preferred embodiment, the present invention encompasses crystalline Sunitinib base designated form XIV characterized by PXRD pattern having peaks at about 14.6 and 21.4±0.2 degrees 2-theta and any 3 peaks at positions selected from the group consisting of 3.5, 9.3, 9.8, 13.9, 15.9, 18.1 and 26.7 deg±0.2 degrees 2-theta.

Crystalline Sunitinib base form XIV can be further characterized by data selected from the group consisting of: a PXRD pattern having peaks at about: 3.5, 9.3, 9.8, 18.1 and 26.7; and a PXRD pattern having double peak at about: 13.9 and 14.6 degrees 2-theta.

Sunitinib base form XIV can be prepared by a process comprising crystallizing sunitinib base from isobutylmethylketone.

The crystallization comprises providing a solution of Sunitinib base and isobutylmethylketone, and precipitating the said crystalline form. Preferably, the said solution is provided by heating the said combination. Preferably the heating is done to a temperature of about 116° C.

Preferably, the precipitation is done by cooling the said solution to obtain a suspension comprising the said crystalline form. Preferably, the cooling is done to about room temperature, more preferably to a temperature of about 20° C.

Preferably, the cooled suspension is maintained at room temperature for about 3 hours.

The process for preparing the said crystalline sunitinib base form XIV may further comprises recovering the crystalline Sunitinib base from the suspension. The recovery may be done for example, by filtering, washing and drying the suspension. The washing can be done with hexane. The drying can be done at room temperature. Preferably the drying is done in air.

In one embodiment, the present invention encompasses crystalline Sunitinib base characterized by data selected from a group consisting of a PXRD pattern having peaks at about 3.9 and 6.0±0.2 degrees 2-theta and any 3 peaks selected from the list consisting of: 3.5, 3.9, 6.0, 9.0, 10.3, 11.8 and 15.0±0.2 degrees 2-theta, a PXRD pattern as depicted in FIG. 18 and combination thereof. This form can be designated form XV.

In a preferred embodiment, the present invention encompasses crystalline Sunitinib base designated form XV characterized by PXRD pattern having peaks at about 3.9 and 6.0±0.2 degrees 2-theta and any 3 peaks at positions selected from the group consisting of: 3.5, 9.0, 10.3, 11.8 and 15.0±0.2 degrees 2-theta.

The above crystalline Sunitinib base designated form XV can be further characterized by data selected from the group consisting of: a powder XRD pattern with peaks at about 3.5, 3.9, 10.3, 11.8 and 15.0±0.2 degrees 2-theta; a powder XRD pattern with peaks at about 3.5, 3.9, 6.0, 11.8 and 15.0±0.2 degrees 2-theta.

Sunitinib base form XV can be prepared by a process comprising crystallizing sunitinib base from a mixture comprising 2-propanol and carbon dioxide.

The crystallization comprises providing a first solution of Sunitinib base in 2-propanol, combining the first solution with gaseous carbon dioxide providing a second solution and precipitating the said crystalline form. Preferably, the first solution is provided by combining Sunitinib base and 2-propanol and heating the said combination. Preferably the heating is done to about reflux temperature.

Further, to the first solution gaseous carbon dioxide is then bubbled.

Preferably, the precipitation is done by cooling the second solution to obtain a suspension comprising the said crystalline form.

Preferably, the cooling is conducted till a temperature of about 25° C. is reached. Preferably, the cooling is conducted for a period of about 30 minutes.

The process for preparing the said crystalline sunitinib base form XV may further comprises recovering the crystalline Sunitinib base from the suspension. The recovery may be done for example, by filtering, washing and drying the suspension. The washing can be done with hexane. The drying can be done at room temperature.

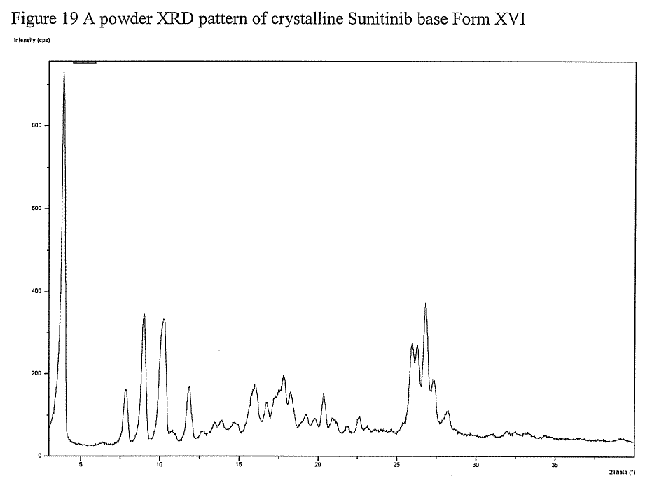

In one embodiment, the present invention encompasses crystalline Sunitinib base characterized by data selected from a group consisting of a PXRD pattern having peaks at about 25.9 and 26.3±0.2 degrees 2-theta and any 3 peaks selected from the list consisting of: 9.0, 10.3, 22.6, 25.9, 26.3 and 27.4±0.2 degrees 2-theta, a PXRD pattern as depicted in FIG. 19 and combination thereof. This form can be designated form XVI.

In a preferred embodiment, the present invention encompasses crystalline Sunitinib base designated form XVI characterized by PXRD pattern having peaks at about 25.9 and 26.3±0.2 degrees 2-theta and any 3 peaks at positions selected from the group consisting of: 9.0, 10.3, 22.6, 25.9, 26.3 and 27.4±0.2 degrees 2-theta.

The above crystalline Sunitinib base designated form XVI can be further characterized by data selected from the group consisting of: a powder XRD pattern with peaks at about 10.3, 22.6, 25.9, 26.3 and 27.4±0.2 degrees 2-theta; and a powder XRD pattern with peaks at about 9.0, 22.6, 25.9, 26.3 and 27.4±0.2 degrees 2-theta.

Sunitinib base form XVI can be prepared by a process comprising crystallizing Sunitinib base from nitromethane.

The crystallization comprises providing a solution of Sunitinib base and nitromethane, and precipitating the said crystalline form.

Preferably, the said solution is provided by combining Sunitinib base and nitromethane and heating the said combination. Preferably the heating is done to about 90° C.

Preferably, the precipitation is done by cooling the said solution to obtain a suspension comprising the said crystalline form. Preferably, the cooling is done to a temperature of about 20° C.

Typically, the suspension is maintained, preferably, for about overnight to increase the yield of the precipitated crystalline form.

The process for preparing the said crystalline sunitinib base form XVI may further comprises recovering the crystalline Sunitinib base from the suspension. The recovery may be done for example, by filtering, washing and drying the suspension. The drying is done at room temperature.

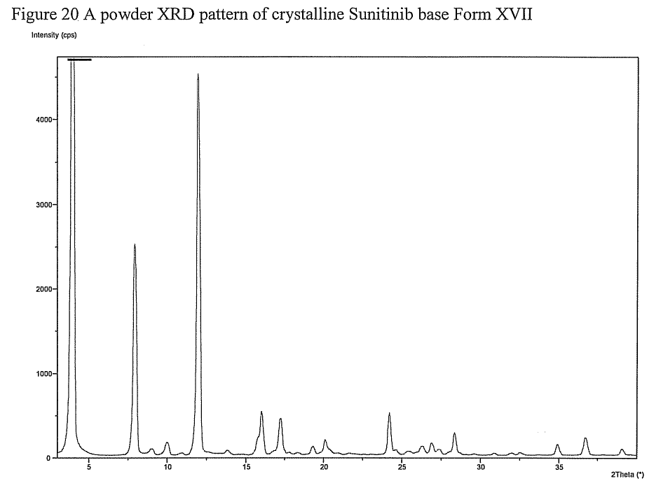

In one embodiment, the present invention encompasses crystalline Sunitinib base characterized by data selected from a group consisting of a PXRD pattern having peaks at about 17.2 and 28.3±0.2 degrees 2-theta and any 3 peaks at positions selected from a group consisting of: 4.0, 7.9, 12.0, 17.2, 24.2, 28.3 and 34.9±0.2 degrees 2-theta, a PXRD pattern as depicted in FIG. 20 and combination thereof. This form can be designated form XVII.

The above crystalline Sunitinib base designated form XVII can be further characterized by data selected from the group consisting of: a powder XRD pattern with peaks at about 12.0, 17.2, 24.2, 28.3 and 34.9±0.2 degrees 2-theta; and a powder XRD pattern with peaks at about 4.0, 7.9, 17.2, 24.2 and 28.3±0.2 degrees 2-theta.

Sunitinib base form XVII can be prepared by a process comprising crystallizing Sunitinib base from methanol.

The crystallization comprises providing a solution of Sunitinib base and methanol, and precipitating the said crystalline form.

Preferably, the said solution is provided by combining Sunitinib base and methanol and heating the said combination. Preferably the heating is done to about 64° C. Preferably, the precipitation is done by cooling the said solution to obtain a suspension comprising the said crystalline form. Preferably, the cooling is done to a temperature of about 20° C.

Typically, the suspension is maintained, preferably, for about overnight to increase the yield of the precipitated crystalline form.

The process for preparing the said crystalline sunitinib base form XVII may further comprises recovering the crystalline Sunitinib base from the suspension. The recovery may be done for example, by filtering, washing and drying the suspension. The drying can be done at room temperature.

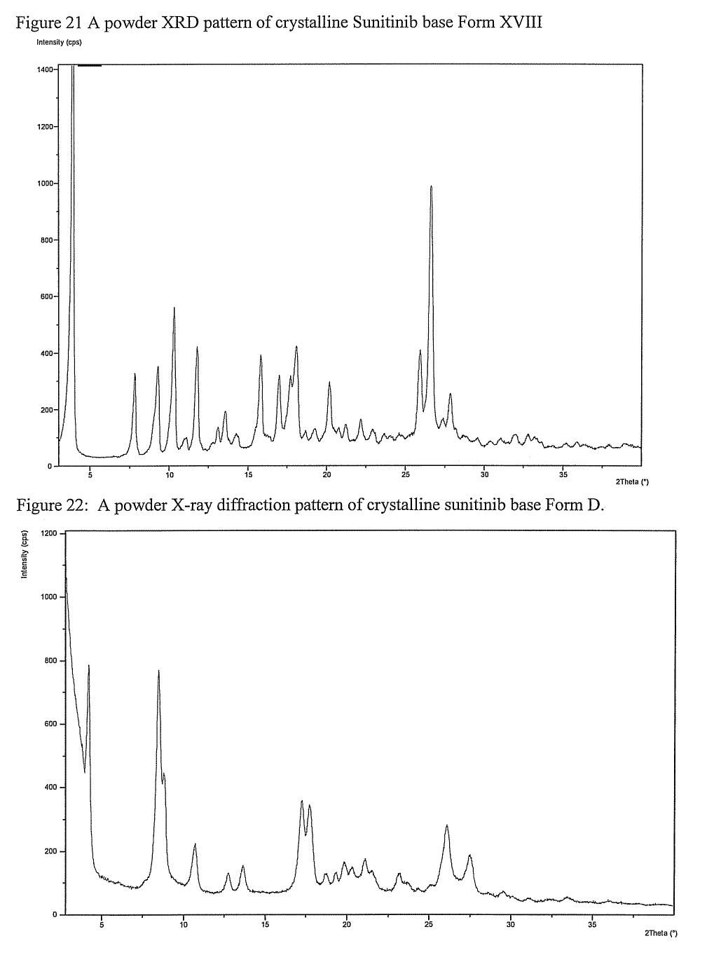

In one embodiment, the present invention encompasses crystalline Sunitinib base characterized by data selected from the group consisting of a PXRD pattern having peaks at about 17.0 and 27.8±0.2 degrees 2-theta and any 3 peaks at positions selected from the group consisting of: 3.9, 10.3, 13.6, 15.8, 17.0, and 18.1±0.2 degrees 2-theta, a PXRD pattern as depicted in FIG. 21 and combination thereof. This form can be designated form XVIII.

The above crystalline Sunitinib base designated form XVIII can be further characterized by data selected from the group consisting of: a powder XRD pattern with peaks at about 3.9, 15.8, 17.0, 18.1 and 27.8±0.2 degrees 2-theta; and a powder XRD pattern with peaks at about 10.3, 13.6, 17.0, 18.1 and 27.8±0.2 degrees 2-theta.

Sunitinib base form XVIII can be prepared by a process comprising crystallizing Sunitinib base from ethanol.

The crystallization comprises providing a solution of Sunitinib base and ethanol, and precipitating the said crystalline form.

Preferably, the said solution is provided by combining Sunitinib base and ethanol and heating the said combination. Preferably the heating is done to about 78° C. Preferably, the precipitation is done by cooling the said solution to obtain a suspension comprising the said crystalline form. Preferably, the cooling is done to a temperature of about 20° C.

Typically, the suspension is maintained, preferably, for about overnight to increase the yield of the precipitated crystalline form.

The process for preparing the said crystalline sunitinib base form XVIII may further comprises recovering the crystalline Sunitinib base from the suspension. The recovery may be done for example, by filtering, washing and drying the suspension. The drying can be done at room temperature.

In one embodiment, the invention encompasses a crystalline form of Sunitinib base characterized by data selected from a group consisting of a PXRD pattern having any 5 peaks at positions selected from the group consisting of: 4.2, 8.5, 10.7, 12.7, 13.6, 17.2, 17.7, 21.1, 26.1 and 27.4±0.2 degrees 2-theta, a PXRD pattern as depicted in FIG. 22 and combination thereof. This crystalline form can be designated Form D.

In a preferred embodiment, the present invention encompasses crystalline Sunitinib base designated form D characterized by a PXRD pattern having peaks at about 3.9 and 4.2±0.2 degrees 2-theta and 3 peaks selected from a list consisting of 8.5, 10.7, 13.6, 17.2, 17.7, 26.1 and 27.4 deg±0.2 degrees 2-theta.

The crystalline Sunitinib base Faun D can be further characterized by data selected from the group consisting of: a PXRD pattern having peaks at about 4.2, 8.5, 10.7, 21.1 and 26.1±0.2 degrees 2-theta; a PXRD pattern having peaks at about 4.2, 8.5, 10.7, 17.7 and 26.1±0.2 degrees 2-theta; and a PXRD pattern having a double peak at about 17.2 and 17.7±0.2 degrees 2-theta. The crystalline Sunitinib base Form D can be prepared by a process comprising heating a suspension comprising sunitinib base, malic acid and toluene.

Preferably, the suspension is provided by combining sunitinib base, malic acid and toluene. Preferably, the said suspension is heated at about reflux temperature. Preferably, the heated suspension is cooled to about room temperature.

The process of preparing the said crystalline form D, may further comprise recovering the Sunitinib base from the heated suspension. The recovery may be done for example, by cooling the heated suspension, filtering the precipitate, washing the precipitate and drying. Preferably, the washing is done with n-hexane. Preferably, the drying is done at about room temperature.

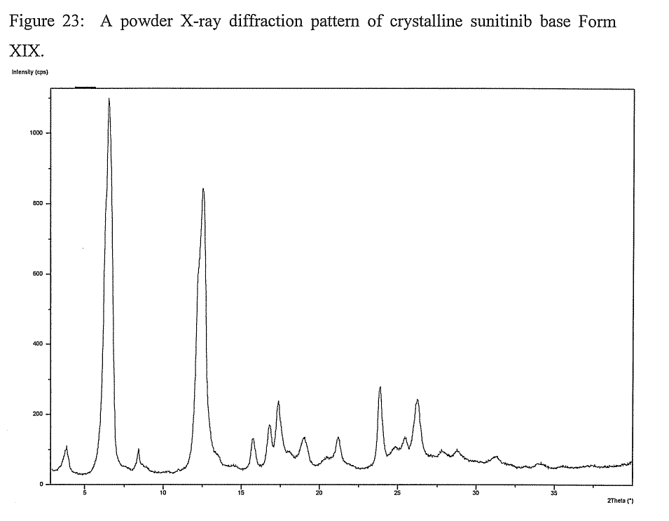

In one embodiment, the present invention encompasses crystalline Sunitinib base characterized by data selected from a group consisting of a PXRD pattern having peaks at about 6.6 and 12.6±0.2 degrees 2-theta and any 3 peaks at positions selected from the group consisting of: 15.8, 16.8, 17.4, 23.8 and 26.1±0.2 degrees 2-theta, a PXRD pattern as depicted in FIG. 23 and combination thereof. This form can be designated form XIX.

The above crystalline Sunitinib base designated form XIX can be further characterized by data selected from the group consisting of: a powder XRD pattern with peaks at about 6.6, 12.6, 15.8, 17.4 and 23.8±0.2 degrees 2-theta; a powder XRD pattern with peaks at about 6.6, 12.6, 16.8, 23.8 and 26.1±0.2 degrees 2-theta, and combination thereof. The crystalline Sunitinib base Form XIX can be prepared by a process comprising heating a suspension comprising sunitinib base, acetic acid and ethanol.

Preferably, the suspension is provided by combining sunitinib base, acetic acid and ethanol. Preferably, the said suspension is heated to about 35° C. to 70° C. More preferably, the said suspension is heated to about 40° C. to 50° C. most preferably, the said suspension is heated to about 35° C. to 70° C. to about 45° C. temperature.

The process of preparing the said crystalline form XIX may further comprises recovering the Sunitinib base from the heated suspension. The recovery may be done for example, by cooling the heated suspension, filtering the precipitate, washing the precipitate and drying. Preferably, the heated suspension is cooled to about 15° C. to −5° C. More preferably, the heated suspension is cooled to about 2° C. to −2° C. Most preferably, the heated suspension is cooled to about 0° C.

Preferably, the washing is done with methyl-THF. Preferably, the drying is done at about 50° C. to about 85° C. More preferably, the drying is done at about 75° C. to about 80° C. Most preferably, the drying is done at about 80° C. Preferably, the drying is conducted for a period of about 8 to about 24 hours. More preferably, the drying is conducted froe a period of about 14 to about 18 hours. Most preferably, the drying is conducted froe a period of about 16 hours.

The present invention further provides the above-described polymorphs of sunitinib base having less than about 15% by weight, of any other form of Sunitinib base, preferably, less than about 10%, more preferably, less than about 5%.

The forms of Sunitinib base disclosed herein can be used to prepare Sunitinib salt, preferably, Sunitinib malate. The present invention provides a process for preparing a Sunitinib salt, comprising preparing the above polymorphs of Sunitinib base and converting them to a Sunitinib salt. Preferably, the polymorphs of Sunitinib base are prepared by the process disclosed herein.

The conversion of Sunitinib base to Sunitinib salt may be done, for example by reacting Sunitinib base with an acid. When the acid is malic acid, the conversion can be done for example according to the process disclosed in US patent application 2003/0069298.

### EXAMPLES

### PXRD

PXRD diffraction was performed on X-Ray powder diffractometer: Philips X′pert Pro powder diffiactometer, CuKα radiation, λ=1.541874 Å. X'Celerator detector active length (2 theta)=2.122 mm, laboratory temperature 22-25° C. Zero background sample-holders.

Prior to analysis the samples were gently ground by means of mortar and pestle in order to obtain a fine powder. The ground sample was adjusted into a cavity of the sample holder and the surface of the sample was smoothed by means of a cover glass.

### DSC

DSC measurements were performed on Differential Scanning Calorimeter DSC823e (Mettler Toledo). Al crucibles 40 μl with PIN were used for sample preparation. Usual weight of sample was 1.5-4 mg.

Program: temperature range 25° C.-250° C., 10° C./min, Nitrogen flow 50 ml/min.

### TGA

TGA measurements were performed on Thermo gravimetric analyzer TGA851e (Mettler Toledo). Alumina crucibles 70 μl were used for sample preparation. Usual weight of sample was 7-13 mg.

Program: temperature range 25° C.-350° C., 10° C./min, Nitrogen flow 50 ml/min.

### KF

KF titrations were performed on automated Karl Fischer titrator TITRANDO 841, software Tiamo 1.1 (Metrohm). Solution used for determination: Hydranal Composite 2 (Riedel de Haen).

### Example 1

### Preparation of Sunitinib Base Sunitinib Base Form

5-Formyl-2,4-dimethyl-1H-pyrrole-3-carboxylic acid, N-(2-diethylaminoethyl)amide (1.3 g), 5-fluoro-1,3-dihydro-indol-2-one (0.7 g), ethanol (10 g) and 2 drops of triethylamine were mixed and heated at reflux)(78° for 3 hours. The suspension was left overnight at room temperature, filtered and the crystalline cake was washed with small amount of ethanol. The product was dried at 70° C. under vacuum for 4 hours to give 1.4 g (72%) of Sunitinib base Form I.

### Example 2

### Preparation of Sunitinib Base Form II

Sunitinib base was dissolved in water (250 g) at about 40° by adjusting the pH to 1.5 with 1N HCl. The solution was extracted with methyl isobutyl ketone (100 g), the phases were separated and to the aqueous phase dimethylacetamide (20 g) was added. Under strong stirring, the solution was adjusted to pH 8.5 by addition of 25% ammonium hydroxide solution. After one hour the suspension was filtered and the cake was rinsed with 200 g of water. The product was dried at 70° under vacuum overnight to give 12.6 g (56%) of Sunitinib base Form II.

### Example 3

### Preparation of Sunitinib Base Form II

14.6 g of Sunitinib base were dissolved in of water (100 g) and 50 g of 1M hydrochloric acid (50 g) at 60° C., the solution was clarified by filtration on paper filter and the filter washed with 50 g of water. Collected solution was basified under stirring at pH 9 with 25% aqueous ammonia. After half hour at room temperature the suspension was filtered and the crystalline cake was washed with 50 g of water. Solid product was dried overnight at 50° under vacuum obtaining 14.3 g, identified by IR to Sunitinib form II.

### Example 4

### Preparation of Sunitinib Base Form III

1.5 g of Sunitinib base was dissolved in 30 g of NMP at 90° C., 30 g of acetone was added and the mixture was left overnight at room temperature under stirring to crystallize, the suspension was filtered and crystals washed with acetone, product was dried at 50° C. under vacuum for 4 hours obtaining 0.8 g.

### Example 5

### Preparation of Sunitinib Base Form III

1.5 g of Sunitinib base was dissolved in 30 g of DMA at 90° C., 30 g of acetone was added and the mixture was left overnight at room temperature under stirring to crystallize, the suspension was filtered and crystals washed with acetone, product was dried at 50° C. under vacuum for 4 hours obtaining 0.9 g.

### Example 6

### Preparation of Sunitinib Base Form IV

Sunitinib base (500 mg) was dissolved in t-butanol (35 ml) at 80° C. The solution was allowed to cool to 25° C., filtered and frozen to −20° C. in refrigenerator. The frozen solid was lyophylized at 1 mBar and 20° C. providing sunitinib base form IV.

### Example 7

### Preparation of Sunitinib Base Form V

Sunitinib base (300 mg) was dissolved in pyridine (1.5 mL) at about 115° C. The solution was allowed to cool down to 25° C. for 5 hours, filtered, washed with tert-butyl methyl ether and dried on air providing sunitinib base form V.

### Example 8

### Preparation of Sunitinib Base Form VI

To sunitinib base Form VII (50 mg) in 25 ml beaker THF (4 ml) and water (0.5 ml) were added and the suspension was heated to the complete dissolution of base (fast, about 1 min). The solution was allowed to cool to RT and allowed to evaporate overnight. Partial evaporation provided a gel, which remained without a noticeable change for 2 days. Than the beaker was placed to the vacuum oven and dried at 50° C. and 1 mBar for 2 h. The dried sample was a solid matter.

### Example 9

### Preparation of Sunitinib Base Form VII

5 g of Sunitinib Base, obtained by reaction of Sunitinib activated carboxylic acid derivative with excess of N,N′-diethylaminoethylamine in tetrahydrofuran, were dissolved with 150 g of water and 50 g of HCl 1M. The solution was washed with methyl isobutyl ketone at 50° C. and then precipitated with ammonia 30% in water to pH 9, the product was filtered and dried in oven under vacuum at 60° C. for 16 hours.

### Example 10

### Preparation of Sunitinib Base Form VIII

Sunitinib base, obtained by reaction of Sunitinib activated carboxylic acid derivative with excess of N,N′-diethylaminoethylamine in 2-Methyltetrahydrofuran, was dissolved in 15 volumes of water (150 ml for 10 g of sunitinib base) at pH 2 (obtained by addition of HCl 1M) at 70° C. The mixture was then cooled to 50° C. and precipitated with the addition of 6 ml of ammonia 30% to pH 8.5, stirred for one hour at the same temperature and filtered at 50° C., washed with 50 ml of water and dried in oven under vacuum for 16 hours at 60° C.

### Example 11

### Preparation of Sunitinib Base Form VIII

Sunitinib base, obtained by reaction of Sunitinib activated carboxylic acid derivative with excess of N,N′-diethylaminoethylamine in 2-Methyltetrahydrofuran, was dissolved in 15 volumes of water (150 ml for 10 g of sunitinib base) at pH 2 (obtained by addition of HCl 1M) at 70° C. The mixture was then cooled to 25° C. and precipitated with the addition of ammonia 30% to pH 8.5 at 25° C., stirred for one hour and filtered at the same temperature, washed with water and dried in oven under vacuum for 16 hours at 60° C.

### Example 12

### Preparation of Sunitinib Base Form IX

Sunitinib base, obtained by reaction of Sunitinib activated carboxylic acid derivative with excess of N,N′-diethylaminoethylamine in 2-Methyltetrahydrofuran, was dissolved in 15 volumes of water (150 ml for 10 g of sunitinib base) at pH 2 (obtained by addition of HCl 1M) at 70° C. The mixture was then cooled to 25° C. and precipitated with the addition of ammonia 30% to pH 8.5 at 25° C., and then heated to 50° C., stirred for one hour at this temperature and filtered at 50° C., washed with water and dried in oven under vacuum for 16 hours at 60° C.

### Example 13

### Preparation of Sunitinib Base Form X

20 g of Sunitinib Base are dissolved with 570 g of water and 190 g of HCl 1M. The solution was washed with methyl isobutyl ketone and then precipitated with ammonia 30% in water to pH 9, the product was filtered and dried in oven under vacuum at 60° C.

The sample contains 15% water by KF.

### Example 14

### Preparation of Sunitinib Base Form XI

Sunitinib base Form X was dried 70° C., 2 h, 1 mBar and 300 mg was added into toluene (10 ml), boiling to 101° C., 10 min, allowed to cool and maintained 3 h at 20° C., filtered, dried in air.

### Example 15

### Preparation of Sunitinib Base Form XII

Sunitinib base Form X was dried 70° C., 2 h, 1 mBar and 150 mg was added into THF (5 ml), boiled to 66° C., 10 min, allowed to evaporate at 20° C. to dryness in an open baker.

### Example 16

### Preparation of Amorphous Sunitinib Base

Sunitinib base Form X was dried 70° C., 2 h, 1 mBar and 150 mg) was added into THF (10 ml), boiled to 66° C., 10 min, allowed to evaporate at 20° C. to dryness in an open baker.

### Example 17

### Preparation of Sunitinib Base Form XIII

Sunitinib base Form X was dried 70 C, 2 h, 1 mBar and 150 mg was added into THF (10 ml), water (1 ml) boiled to 66° C., 10 min, allowed to evaporate at 20° C. to dryness in an open baker.

### Example 18

### Preparation of Sunitinib Base Form XIV

Sunitinib base Form X was dried 70° C., 2 h, 1 mBar and 150 mg was added into isobutylmethylketon (20 ml), boiled to 116° C., 30 min, allowed to cool to RT, maintained 3 h, filtered, washed with hexane, dried in air.

### Example 19

### Conversion of Sunitinib to Sunitinib Malate (According to Example 1, Preparation A of U.S. publication No. 2003/0069298)

Preparation of the Anhydrous Crystal Form I of the L-Malic Acid Salt of N-[2-(Diethylamino) ethyl]-5-[(5-fluoro-1,2-dihydro-2-oxo-3H-indol-3-ylidene) methyl]-2,4-dimethyl-1H-pyrrole-3-carboxamide.

Preparation a: N[2-(Diethylamino)ethyl′-5-[(5-fluoro-1,2-dihydro-2-1-oxo-3H-indol-3-ylidene)methyl]-2,4-dimethyl-1H-pyrrole-3-carboxamide (130 mg, 0.326 mMol) was added to 20 mL methanol, and the mixture was stirred. L-malic acid (47.2 mg, 0.352 mMol) was added, resulting in rapid dissolution of all the solids. The methanol was removed under reduced pressure to produce a poorly crystalline orange solid. Acetonitrile (5 mL) was added, and the slurry was stirred and heated for about 10 minutes. Stirring was continued while the slurry was allowed to cool to room temperature. The crystals were filtered and dried, resulting in 149 mg of solids (86% yield).

### Example 20

### Preparation of Sunitinib Base Form XV

Sunitinib base (200 mg) was dissolved in 2-propanol (17 ml) by heating 5 ml to reflux temperature. Then carbon dioxide was bubbled to the solution. The solution was allowed to cool to a temperature of about 25° C. and sunitinib started to crystallize. Sample was filtered, washed with hexane (10 ml) and dried in air, resulting in 78 mg of solids.

### Example 21

### Preparation of Sunitinib Base Form XVI

Sunitinib base (Form D, 200 mg) was dissolved in nitromethane (8 ml) by heating to 90 C. The solution was allowed to cool to 20° C. and stand overnight. Crystals were collected by filtration and dried in air.

### Example 22

### Preparation of Sunitinib Base Form XVII

Sunitinib base (Form D, 200 mg) was dissolved in methanol (6 ml) by heating to 64° C. The solution was allowed to cool to 20° C. and stand overnight. Crystals were collected by filtration and dried in air.

### Example 23

### Preparation of Sunitinib Base Form XVIII

Sunitinib base (Form D, 200 mg) was dissolved in ethanol (7 ml) by heating to 78° C. The solution was allowed to cool to 20° C. and stand overnight. Crystals were collected by filtration and dried in air.

### Example 24

### Preparation of Sunitinib Base Via 5-(5-fluoro-2-oxo-1,2-dihydro-indol-3Z-ylidenemethyl)-2,4-dimethyl-1H-pyrrole-3-carbonyl chloride

31.2 g of 5-(5-fluoro-2-oxo-1,2-dihydro-indol-3Z-ylidenemethyl)-2,4-dimethyl-1H-pyrrole-3-carboxylic acid, were refluxed under stirring for 4 hours in one liter flask with 310 g of toluene, 15 g of thionyl chloride and 1 g of dimethylformamide. The stirred suspension was cooled at room temperature for 2 hours and filtered; the cake was washed with 50 g of toluene and dried at 50° under vacuum overnight. Yield was 32.4 g (97.8%) of a compound corresponding by NMR and MS to the expected structure.

20 g of diethylendiamine were dissolved in one liter flask with 300 g of tetrahydrofuran; about 200 g of solvent were distilled away at 50° under vacuum. 20 g of 5-(5-fluoro-2-oxo-1,2-dihydro-indol-3Z-ylidenemethyl)-2,4-dimethyl-1H-pyrrole-3-carbonyl chloride, prepared as above, were added under stirring and solution obtained was left for one hour to react without more heating.

300 g of water were added and the suspension was evaporated at 50° under vacuum to eliminate most of organic solvent.

After stirring 2 hours at room temperature the suspension was filtered, washed with 100 g of water and dried at 50° under vacuum overnight, obtaining 23.5 g of crude Sunitinib.

### Purification

Crude material was dissolved with 560 g of water and 190 g of 1 M Hydrochloric acid, extracted with 200 g of methyl-isobutyl ketone.

Clarified aqueous phase was basified under stirring with concentrated aqueous ammonia to pH 8.5 and after 2 hours the suspension was filtered and crystals were washed with 100 g of water.

Product was dried at 50° under vacuum overnight obtaining 20.5 (82% yield) of sunitinib.

### Example 25

### Preparation of Sunitinib Base Via Sunitinib Carboxylic Acid Derivative

In a 500 ml reactor, 15.0 g. of Sunitinib carboxylic acid derivative were suspended into 300 ml of toluene (ratio 20/1.0 v/w starting material) under vigorous stirring at room temperature.

0.755 g of dimethylformamide (ratio 0.2/1.0 w/w) were added to the mixture. The temperature was set at 70° C. and at this temperature, 5.1 g of thionyl chloride (ratio 1.4/1.0 w/w) were dropped in a range of sixty minutes.

The reaction was kept at 70° C. for 7 hours under stirring.

Then 140 ml of solvent were distilled to remove excess of thionyl chloride from the suspension and the reaction filtered on gooch P3 washing with 3v/w of toluene. The wet solid (sunitinib acyl chloride derivative) was re-loaded into the reactor and 300 ml Methyl-tetrahydrofuran was loaded and stirred. Then the reaction mixture was heated to 70° C. and 6.35 g of 2-diethylamino-ethylamine (ratio 1.1/1.0 w/w starting material) were dropped in five minutes at 70° C. After one hour the reaction was completed and 150 ml of water and HCl 2N until pH 2 were added to the suspension.

A following filtration of the mixture using a decalite pad was done to obtain a clarified phase. The two phases were separated at 50° C. and the organic phase discarded. The aqueous phase was washed once more with 300 ml of Methyl-tetrahydrofuran at 50° C. under stirring. The two phases were separated again and the organic phase discarded.

The aqueous phase was then basified to pH 8.5 with 5% ammonia solution at 50° C.

After one hour stirring, the suspension was filtered on gooch P3 and the wet solid dried at 60° C. under vacuum overnight.

15.9 g. of sunitinib base were obtained with a purity of NLT 99.5% by HPLC

### Example 26

### Preparation of Sunitinib Base Form D

Sunitinib base (Form II, 300 mg), L-malic acid (101 mg) and toluene (10 ml) were heated to reflux 10 min. The suspension was allowed to cool to room temperature, filtered, washed with n-hexane and dried in air.

### Example 27

### Preparation of Sunitinib Base Form XIX

2 g of sunitinib base were suspended in 40 ml of ethanol at 25° C., then 0.3 g of acetic acid were added, the suspension heated to 45° C. with partial dissolution. It was cooled to 0° C. in 3 hrs, it was left stirring for 1 hour at 0° C., filtered and washed with Me-THF with 4% of purified water. Dried at 80° under vacuum for 16 hours.

1. A crystalline Sunitinib base characterized by data selected from the group consisting of a PXRD pattern having any 5 peaks selected from the list consisting of: 3.8, 7.6, 8.5, 9.5, 10.4, 11.4, 16.5, 17.8, 20.6, and 27.0 deg±0.2 degrees 2-theta, a PXRD pattern as depicted in FIG. 10 and combination thereof.

2. The crystalline of sunitinib base of claim 1, characterized by a PXRD pattern having any 5 peaks selected from the list consisting of: 3.8, 7.6, 8.5, 9.5, 10.4, 11.4, 16.5, 17.8, 20.6, and 27.0 deg±0.2 degrees 2-theta.

3. The crystalline of sunitinib base of claim 1, characterized by a PXRD pattern as depicted in FIG. 10.

4. The crystalline of sunitinib base of claim 1, characterized by PXRD pattern having peaks at about 7.6 and 16.5±0.2 degrees 2-theta and any 3 peaks at positions selected from the group consisting of 3.8, 8.5, 9.5, 11.4, 17.8, 20.6 and 27.0 deg±0.2 degrees 2-theta.

5. The crystalline of sunitinib base of claim 4, further characterized by a PXRD pattern having peaks at about: 3.8, 7.6, 9.5, 16.5 and 20.6±0.2 degrees 2-theta.

6. The crystalline of sunitinib base of claim 4, further characterized by a PXRD pattern having peaks at about: 3.8, 7.6, 9.5, 17.8 and 27.0±0.2 degrees 2-theta.

7. The crystalline of sunitinib base of claim 4, further characterized by a PXRD pattern having peaks at about 3.8, 7.6, 9.5, 10.4 and 11.4±0.2 degrees 2-theta.

8. A process for preparing a crystalline sunitinib base characterized by data selected from the group consisting of a PXRD pattern having any 5 peaks selected from the list consisting of: 3.8, 7.6, 8.5, 9.5, 10.4, 11.4, 16.5, 17.8, 20.6, and 27.0 deg±0.2 degrees 2-theta, a PXRD pattern as depicted in FIG. 10 and combination thereof, comprising a) providing a mixture of Sunitinib base and 2-methyltetrahydrofuran, b) combining the mixture of step a) with acidic water to obtain a solution, c) cooling the said solution, and d) precipitating the said crystalline form by adding a base to the cooled solution of step c).

9. The process of claim 8, wherein the mixture of Sunitinib base in 2-methyltetrahydrofuran is provided by reacting Sunitinib activated carboxylic acid derivative with N,N-diethylaminoethylamine in 2-methyltetrahydrofuran.

10. The process of claim 8, further comprise recovering the crystalline sunitinib base.

11. A crystalline Sunitinib base characterized by data selected from the group consisting of a PXRD pattern having any 5 peaks at positions selected from the group consisting of: 4.2, 8.5, 10.7, 12.7, 13.6, 17.2, 17.7, 21.1, 26.1 and 27.4±0.2 degrees 2-theta, a PXRD pattern as depicted in FIG. 22 and combination thereof.

12. The crystalline Sunitinib base of claim 11, characterized by a PXRD pattern having any 5 peaks at positions selected from the group consisting of: 4.2, 8.5, 10.7, 12.7, 13.6, 17.2, 17.7, 21.1, 26.1 and 27.4±0.2 degrees 2-theta.

13. The crystalline Sunitinib base of claim 11, characterized by a PXRD pattern as depicted in FIG. 22.

14. The crystalline Sunitinib base of claim 11, characterized by a PXRD pattern having peaks at about 3.9 and 4.2±0.2 degrees 2-theta and 3 peaks selected from a list consisting of 8.5, 10.7, 13.6, 17.2, 17.7, 26.1 and 27.4 deg±0.2 degrees 2-theta.

15. The crystalline Sunitinib base of claim 14, further characterized by a PXRD pattern having peaks at about 4.2, 8.5, 10.7, 21.1 and 26.1±0.2 degrees 2-theta.

16. The crystalline Sunitinib base of claim 14, further characterized by a PXRD pattern having peaks at about 4.2, 8.5, 10.7, 17.7 and 26.1±0.2 degrees 2-theta.

17. The crystalline Sunitinib base of claim 14, further characterized by a PXRD pattern having a double peak at about 17.2 and 17.7±0.2 degrees 2-theta.

18. A process for preparing a crystalline Sunitinib base characterized by data selected from the group consisting of: a PXRD pattern having peaks at about 4.2, 8.5, 10.7, 21.1 and 26.1±0.2 degrees 2-theta; a PXRD pattern having peaks at about 4.2, 8.5, 10.7, 17.7 and 26.1±0.2 degrees 2-theta; and a PXRD pattern having a double peak at about 17.2 and 17.7±0.2 degrees 2-theta, comprising heating a suspension comprising sunitinib base, malic acid and toluene.

19. The process of claim 18, further comprising recovering the Sunitinib base from the heated suspension.

20. A polymorph of Sunitinib base selected from the group consisting of: a crystalline Sunitinib base characterized by data selected from the group consisting of a PXRD pattern having any 5 peaks selected from the list consisting of: 3.8, 7.8, 9.0, 10.2, 11.8, 15.8, 17.9, 20.3, 26.1 and 26.8 deg±0.2 degrees 2-theta, a PXRD pattern as depicted in FIG. 2 and combination thereof; a crystalline Sunitinib base characterized by data selected from a group consisting of a PXRD pattern having any 5 peaks selected from the list consisting of: 5.0, 10.0, 13.0, 14.7, 16.0, 20.1, 21.9, 25.7, 26.6 and 28.3 deg±0.2 degrees 2-theta, a PXRD pattern as depicted in FIG. 5 and combination thereof; a crystalline Sunitinib base characterized by data selected from a group consisting of a PXRD pattern having any 5 peaks selected from the list consisting of: 7.6, 9.3, 12.1, 15.2, 16.3, 19.2, 24.2, 25.5, 26.2 and 28.2 deg±0.2 degrees 2-theta, a PXRD pattern as depicted in FIG. 6; a crystalline Sunitinib base characterized by data selected from a group consisting of a PXRD pattern having any 5 peaks selected from the list consisting of: 3.5, 5.9, 6.5, 8.3, 10.5, 11.3, 14.1, 17.9, 20.7, 26.0 and 27.9 deg±0.2 degrees 2-theta, a PXRD pattern as depicted in FIG. 7 and combination thereof; a crystalline Sunitinib base characterized by data selected from a group consisting of a PXRD pattern having any 5 peaks selected from the list consisting of: 8.2, 10.9, 13.2, 13.6, 16.1, 18.8, 19.2, 20.9, 23.5, 26.1 and 29.6 deg±0.2 degrees 2-theta, a PXRD pattern as depicted in FIG. 8 and combination thereof; a crystalline Sunitinib base characterized by data selected from a group consisting of a PXRD pattern having any 5 peaks selected from the list consisting of: 3.9, 7.8, 8.9, 9.2, 11.7, 13.9, 15.4, 16.0, 17.9, 20.4, 26.7 and 27.8 deg±0.2 degrees 2-theta, a PXRD pattern as depicted in FIG. 9 and combination thereof; a crystalline Sunitinib base characterized by data selected from a group consisting of a PXRD pattern having any 5 peaks selected from the list consisting of: 3.8, 7.6, 8.9, 9.3, 10.4, 17.8, 20.5, 21.7, 26.2, and 26.9 deg±0.2 degrees 2-theta, a PXRD pattern as depicted in FIG. 11 and combination thereof; a crystalline Sunitinib base characterized by data selected from a group consisting of a PXRD pattern having any 5 peaks selected from the list consisting of: 3.9, 7.8, 9.1, 10.1, 11.6, 14.3, 15.9, 18.2, 20.4, 23.1, 23.9 and 27.0 deg±0.2 degrees 2-theta, a PXRD pattern as depicted in FIG. 12 and combination thereof; a crystalline Sunitinib base characterized by data selected from a group consisting of a PXRD pattern having any 5 peaks selected from the list consisting of: 3.8, 4.2, 8.5, 10.7, 12.7, 13.6, 17.2, 17.7, 26.1 and 27.5 deg±0.2 degrees 2-theta, a PXRD pattern as depicted in FIG. 13 and combination thereof; a crystalline Sunitinib base characterized by data selected from a group consisting of a PXRD pattern having any 5 peaks selected from the list consisting of: 5.3, 8.3, 10.6, 11.0, 11.7, 15.0, 15.7, 16.0 and 27.0 deg±0.2 degrees 2-theta, a PXRD pattern as depicted in FIG. 14 and combination thereof; an amorphous Sunitinib base characterized by a PXRD pattern having broad diffraction peak, as depicted in FIG. 15; a crystalline Sunitinib base characterized by data selected from a group consisting of a PXRD pattern having any 5 peaks selected from the list consisting of: 8.3, 8.5, 10.5, 13.1, 14.9, 16.0, 18.1, 18.7, 19.2, 20.8, 23.4, 26.1 and 29.5 deg±0.2 degrees 2-theta, a PXRD pattern as depicted in FIG. 16 and combination thereof; a crystalline Sunitinib base characterized by data selected from a group consisting of a PXRD pattern having any 5 peaks selected from the list consisting of: 3.5, 9.3, 9.8, 11.7, 13.9, 14.6, 15.9, 18.1, 21.4 and 26.7 deg±0.2 degrees 2-theta, a PXRD pattern as depicted in FIG. 17 and combination thereof; a crystalline Sunitinib base characterized by data selected from a group consisting of a PXRD pattern having peaks at about 3.9 and 6.0±0.2 degrees 2-theta and any 3 peaks selected from the list consisting of: 3.5, 3.9, 6.0, 9.0, 10.3, 11.8 and 15.0±0.2 degrees 2-theta, a PXRD pattern as depicted in FIG. 18 and combination thereof; a crystalline Sunitinib base characterized by data selected from a group consisting of a PXRD pattern having peaks at about 25.9 and 26.3±0.2 degrees 2-theta and any 3 peaks selected from the list consisting of: 9.0, 10.3, 22.6, 25.9, 26.3 and 27.4±0.2 degrees 2-theta, a PXRD pattern as depicted in FIG. 19 and combination thereof; a crystalline Sunitinib base characterized by data selected from a group consisting of a PXRD pattern having peaks at about 17.2 and 28.3±0.2 degrees 2-theta and any 3 peaks at positions selected from a group consisting of: 4.0, 7.9, 12.0, 17.2, 24.2, 28.3 and 34.9±0.2 degrees 2-theta, a PXRD pattern as depicted in FIG. 20 and combination thereof; a crystalline Sunitinib base characterized by data selected from the group consisting of a PXRD pattern having peaks at about 17.0 and 27.8±0.2 degrees 2-theta and any 3 peaks at positions selected from the group consisting of: 3.9, 10.3, 13.6, 15.8, 17.0, and 18.1±0.2 degrees 2-theta, a PXRD pattern as depicted in FIG. 21 and combination thereof; and a crystalline Sunitinib base characterized by data selected from a group consisting of a PXRD pattern having peaks at about 6.6 and 12.6±0.2 degrees 2-theta and any 3 peaks at positions selected from the group consisting of: 15.8, 16.8, 17.4, 23.8 and 26.1±0.2 degrees 2-theta, a PXRD pattern as depicted in FIG. 23 and combination thereof.

21. (canceled)

22. (canceled)

23. (canceled)

24. A method for the preparation of a Sunitinib salt, comprising converting the crystalline Sunitinib base of claim 1 or the polymorph of Sunitinib base of claim 20 to the Sunitinib salt.

25. A method for preparing Sunitinib malate, comprising preparing the crystalline Sunitinib base of claim 1 or the polymorph of Sunitinib base of claim 20, and converting crystalline Sunitinib base or the polymorph of Sunitinib base to Sunitinib malate.
